(19) United States
(12) Patent Application Publication
(10) Pub. No.: US 2006/0067930 A1
(43) Pub. Date: Mar. 30, 2006

(54) Polypeptide variants with altered effector function

(75) Inventors: Camellia W. Adams, San Jose, CA (US); Samantha Lien, San Francisco, CA (US); Henry B. Lowman, El Granada, CA (US); Jonathan S. Marvin, New York, NY (US); Yu-Ju G. Meng, Albany, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/208,422

(22) Filed: Aug. 19, 2005

Related U.S. Application Data
(60) Provisional application No. 60/603,057, filed on Aug. 19, 2004.

(51) Int. Cl. A61K 39/395; C07K 16/44

(52) U.S. Cl. 424/133.1; 530/387.3

(57) ABSTRACT

The invention provides polypeptides having IgG Fc regions with amino acid modifications that result in the polypeptides exhibiting altered Fc effector functions.

This application claims benefit of provisional application Ser. No. 60/603,057, filed on Aug. 19, 2004, which application is incorporated herein by reference in their entirety.

### FIELD OF THE INVENTION

The present invention concerns polypeptides comprising a variant Fc region. More particularly, the present invention concerns Fc region-containing polypeptides that have altered effector function as a consequence of one or more amino acid modifications in the Fc region thereof.

### BACKGROUND OF THE INVENTION

Antibodies are proteins that exhibit binding specificity to a specific antigen. Native antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains.

The term “variable” refers to the fact that certain portions of the variable domains differ extensively in sequence among antibodies and are responsible for the binding specificity of each particular antibody for its particular antigen. However, the variability is not evenly distributed through the variable domains of antibodies. It is concentrated in three segments called complementarity determining regions (CDRs) both in the light chain and the heavy chain variable domains. The more highly conserved portions of the variable domains are called the framework regions (FRs). The variable domains of native heavy and light chains each comprise four FRs, largely adopting a β-sheet configuration, connected by three CDRs, which form loops connecting, and in some cases forming part of, the β-sheet structure. The CDRs in each chain are held together in close proximity by the FRs and, with the CDRs from the other chain, contribute to the formation of the antigen binding site of antibodies (see Kabat et al., Sequences of Proteins of Immunological Interest, 5th Ed. Public Health Service, National Institutes of Health, Bethesda, Md. (1991)).

The constant domains are not involved directly in binding an antibody to an antigen, but exhibit various effector functions. Depending on the amino acid sequence of the constant region of their heavy chains, antibodies or immunoglobulins can be assigned to different classes. There are five major classes of immunoglobulins: IgA, IgD, IgE, IgG and IgM, and several of these may be further divided into subclasses (isotypes), e.g. IgG1, IgG2, IgG3, and IgG4; IgA1 and IgA2. The heavy chain constant regions that correspond to the different classes of immunoglobulins are called α, δ, ε, γ, and μ, respectively. Of the human immunoglobulin classes, only human IgG1, IgG2, IgG3 and IgM are known to activate complement, and human IgG1 and IgG3 mediate ADCC more effectively than IgG2 and IgG4.

A schematic representation of the native IgG1 structure is shown in FIG. 1A, where the various portions of the native antibody molecule are indicated. Papain digestion of antibodies produces two identical antigen binding fragments, called Fab fragments, each with a single antigen binding site, and a residual “Fc” fragment, whose name reflects its ability to crystallize readily. The crystal structure of the human IgG Fc region has been determined (Deisenhofer, Biochemistry 20:2361-2370 (1981)). In human IgG molecules, the Fc region is generated by papain cleavage N-terminal to Cys 226. The Fc region is central to the effector functions of antibodies.

Antibody Effector Functions

The effector functions mediated by the antibody Fc region can be divided into two categories: (1) effector functions that operate after the binding of antibody to an antigen (these functions involve the participation of the complement cascade or Fc receptor (FcR)-bearing cells); and (2) effector functions that operate independently of antigen binding (these functions confer persistence in the circulation and the ability to be transferred across cellular barriers by transcytosis). Ward and Ghetie, Therapeutic Immunology 2:77-94 (1995).

While binding of an antibody to the requisite antigen has a neutralizing effect that might prevent the binding of a foreign antigen to its endogenous target (e.g. receptor or ligand), binding alone may not remove the foreign antigen. To be efficient in removing and/or destructing foreign antigens, an antibody should be endowed with both high affinity binding to its antigen, and efficient effector functions.

The interaction of antibodies and antibody-antigen complexes with cells of the immune system effects a variety of responses, including antibody-dependent cell-mediated cytotoxicity (ADCC) and complement dependent cytotoxicity (CDC) (reviewed in Daëron, Annu. Rev. Immunol. 15:203-234 (1997); Ward and Ghetie, Therapeutic Immunol. 2:77-94 (1995); as well as Ravetch and Kinet, Annu. Rev. Immunol. 9:457-492 (1991)).

Several antibody effector functions are mediated by Fc receptors (FcRs), which bind the Fc region of an antibody. FcRs are defined by their specificity for immunoglobulin isotypes; Fc receptors for IgG antibodies are referred to as FcγR, for IgE as FcεR, for IgA as FcαR and so on. Three subclasses of FcγR have been identified: FcγRI (CD64), FcγRII (CD32) and FcγRIII (CD16). Because each FcγR subclass is encoded by two or three genes, and alternative RNA splicing leads to multiple transcripts, a broad diversity in FcγR isoforms exists. The three genes encoding the FcγRI subclass (FcγRIA, FcγRIB and FcγRIC) are clustered in region 1q21.1 of the long arm of chromosome 1; the genes encoding FcγRII isoforms (FcγRIIA, FcγRIIB and FcγRIIC) and the two genes encoding FcγRIII (FcγRIIIA and FcγRIIIB) are all clustered in region 1q22. These different FcR subtypes are expressed on different cell types (reviewed in Ravetch and Kinet, Annu. Rev. Immunol. 9:457-492 (1991)). For example, in humans, FcγRIIIB is found only on neutrophils, whereas FcγRIIIA is found on macrophages, monocytes, natural killer (NK) cells, and a subpopulation of T-cells.

Structurally, the FcγR are all members of the immunoglobulin superfamily, having an IgG-binding α-chain with an extracellular portion comprised of either two (FcγRI and FcγRIII) or three (FcγRI ) Ig-like domains. In addition, FcγRI and FcγRIII have accessory protein chains (γ,ζ) associated with the α-chain which function in signal transduction. The receptors are also distinguished by their affinity for IgG. FcγRI exhibits a high affinity for IgG, Kₐ=10⁸-10⁹M⁻¹ (Ravetch et al. Ann. Rev. Immunol. 19:275-290 (2001)) and can bind monomeric IgG. In contrast FcγRII and FcγRIII show a relatively weaker affinity for monomeric IgG Kₐ≦10⁷M⁻¹ (Ravetch et al. Ann. Rev. Immunol. 19:275-290 (2001)), and hence only interact effectively with multimeric immune complexes. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review in Daëon, Annu. Rev. Immunol. 15:203-234 (1997)). NK cells carry only FcγRIIIA and binding of antibodies to FcγRIIIA leads to ADCC activity by the NK cells.

Allelic variants of several of the human FcγR have been found in the human population. These allelic variant forms have been shown to exhibit differences in binding of human and murine IgG and a number of association studies have correlated clinical outcomes with the presence of specific allelic forms (reviewed in Lehrnbecher et al. Blood 94(12):4220-4232 (1999)). Several studies have investigated two forms of FcγRIIA, R131 and H131, and their association with clinical outcomes (Hatta et al. Genes and Immunity 1:53-60 (1999); Yap et al. Lupus 8:305-310 (1999); and Lorenz et al. European J. Immunogenetics 22:397-401 (1995)). Two allelic forms of FcγRIIIA, F158 and V158, are only now being investigated (Lehrnbecher et al., supra; and Wu et al. J. Clin. Invest. 100(5): 1059-1070 (1997)). The FcγRIIIA(Vall58) allotype interacts with human IgG better than the FcγRIIIA(Phel58) allotype (Shields et al. J. BioL Chem. 276: 6591-6604 (2001); Koene et al. Blood 90:1109-1114 (1997); and Wu et al. J. Clin. Invest. 100: 1059-1070 (1997)).

The binding site on human and murine antibodies for FcγR have been previously mapped to the so-called “lower hinge region” consisting of residues 233-239 (EU index numbering as in Kabat et al., Sequences of Proteins of Immunological Interest, 5th Ed. Public Health Service, National Institutes of Health, Bethesda, Md. (1991)). Woof et al. Molec. Immunol. 23:319-330 (1986); Duncan et al. Nature 332:563 (1988); Canfield and Morrison, J. Exp. Med. 173:1483-1491 (1991); Chappel et al., Proc. Natl. Acad. Sci USA 88:9036-9040 (1991). Of residues 233-239, P238 and S239 have been cited as possibly being involved in binding.

Other previously cited areas possibly involved in binding to FcγR are: G316-K338 (human IgG) for human FcγRI (by sequence comparison only; no substitution mutants were evaluated) (Woof et al. Molec. Immunol. 23:319-330 (1986)); K274-R301 (human IgG1) for human FcγRIII (based on peptides) (Sarmay et al. Molec. Immunol. 21:43-51 (1984)); Y407-R416 (human IgG) for human FcγRIII (based on peptides) (Gergely et al. Biochem. Soc. Trans. 12:739-743 (1984)); as well as N297 and E318 (murine IgG2b) for murine FcγRII (Lund et al., Molec. Immunol. 29:53-59 (1992)). See also Armour et al. Eur. J. Immunol. 29: 2613-2624 (1999).

Presta in U.S. Pat. No. 6,737,056 describes polypeptide variants with improved or diminished binding to FcRs. See, also, Shields et al. J. Biol. Chem. 9(2): 6591-6604 (2001). Variant Fcs that bind FcγR are also described in WO 2004/063351.

C1q and two serine proteases, C1r and C1s, form the complex C1, the first component of the complement dependent cytotoxicity (CDC) pathway. Clq is a hexavalent molecule with a molecular weight of approximately 460,000 and a structure likened to a bouquet of tulips in which six collagenous “stalks” are connected to six globular head regions. Burton and Woof, Advances in Immunol. 51:1-84 (1992). To activate the complement cascade, it is necessary for C1q to bind to at least two molecules of IgG1, IgG2, or IgG3 (the consensus is that IgG4 does not activate complement), but only one molecule of IgM, attached to the antigenic target. Ward and Ghetie, Therapeutic Immunology 2:77-94 (1995) at page 80.

Based upon the results of chemical modifications and crystallographic studies, Burton et al. Nature, 288:338-344 (1980) proposed that the binding site for the complement subcomponent C1q on IgG involves the last two (C-terminal) β-strands of the CH2 domain. Burton later suggested (Molec. Immunol., 22(3):161-206 (1985)) that the region comprising amino acid residues 318 to 337 might be involved in complement fixation.

Duncan and Winter Nature 332:738-40 (1988), using site directed mutagenesis, reported that Glu318, Lys320 and Lys322 form the binding site to C1q. The data of Duncan and Winter were generated by testing the binding of a mouse IgG2b isotype to guinea pig C1q. The role of Glu3l8, Lys320 and Lys322 residues in the binding of C1q was confirmed by the ability of a short synthetic peptide containing these residues to inhibit complement mediated lysis. Similar results are disclosed in U.S. Pat. No. 5,648,260 issued on Jul. 15, 1997, and U.S. Pat. No. 5,624,821 issued on Apr. 29, 1997.

The residue Pro331 has been implicated in C1q binding by analysis of the ability of human IgG subclasses to carry out complement mediated cell lysis. Mutation of Ser331 to Pro331 in IgG4 conferred the ability to activate complement. (Tao et al., J. Exp. Med., 178:661-667 (1993); Brekke et al., Eur. J. Immunol., 24:2542-47 (1994)).

From the comparison of the data of the Winter group, and the Tao et al. and Brekke et al. papers, Ward and Ghetie concluded in their review article that there are at least two different regions involved in the binding of C1q: one on the β-strand of the CH2 domain bearing the Glu318, Lys320 and Lys322 residues, and the other on a turn located in close proximity to the same β-strand, and containing a key amino acid residue at position 331.

Other reports suggested that human IgG1 residues Lys235, and Gly237, located in the lower hinge region, play a critical role in complement fixation and activation. Xu et al., J. Immunol. 150: 152A (Abstract) (1993). WO94/29351 published Dec. 22, 1994 reports that amino acid residues necessary for C1q and FcR binding of human IgG1 are located in the N-terminal region of the CH2 domain, i.e. residues 231 to 238.

It has further been proposed that the ability of IgG to bind C1q and activate the complement cascade also depends on the presence, absence or modification of the carbohydrate moiety positioned between the two CH2 domains (which is normally anchored at Asn297). Ward and Ghetie, Therapeutic Immunology 2:77-94 (1995) at page 81.

Polypeptide variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

Another type of Fc receptor is the neonatal Fc receptor (FcRn). FcRn is structurally similar to major histocompatibility complex (MHC) and consists of an α-chain noncovalently bound to β-microglobulin. The multiple functions of the neonatal Fc receptor FcRn are reviewed in Ghetie and Ward (2000) Annu. Rev. Immunol. 18, 739-766. The FcRn plays a key role in IgG homeostasis based on a pH-dependent interaction with the antibody Fc region (Ghetie and Ward (2000) Annu Rev Immunol 18, 739-766; Ghetie and Ward (1997) Immunol Today 18,592-598). Increasing the affinity of the Fc-FcRn complex at pH 6 while retaining low affinity at pH 7.4 has been shown to increase antibody half-life (Hinton et al. (2004) J Biol Chem 279, 6213-6216). FcRn plays a role in the passive delivery of immunoglobulin IgGs from mother to young and the regulation of serum IgG levels. FcRn acts as a salvage receptor, binding and transporting pinocytosed IgGs in intact form both within and across cells, and rescuing them from a default degradative pathway, as illustrated in FIG. 6. Although the mechanisms responsible for salvaging IgGs are still unclear, it is thought that unbound IgGs are directed toward proteolysis in lysosomes, whereas bound IgGs are recycled to the surface of the cells and released. This control takes place within the endothelial cells located throughout adult tissues. FcRn is expressed in at least the liver, mammary gland, and adult intestine.

FcRn binds to IgG; the FcRn-IgG interaction has been studied extensively and appears to involve residues at the CH2, CH3 domain interface of the Fc region of IgG. These residues interact with residues primarily located in the α2 domain of FcRn

Ghetie et al. in Nature Biotechnology 15: 637-640 (1997) reported random mutagenesis of Thr252, Thr254, and Thr256 in murine Fcγ1, residues that are in proximity to the FcRn-IgG interaction site, to study the effect on the serum half-lives of these variant hinge-Fc fragments. The mutant with the highest affinity for murine FcRn has a longer half-life than the wild-type fragment despite its lower off-rate from FcRn at pH 7.4.

In previous studies, extensive alanine-scanning by Presta and colleagues (Shields et al., J. Biol. Chem. 276: 6591-6604 (2001); Presta U.S. Pat. No. 6,737,056) identified three Fc variants, N434A, E380A, and T307A, that increase the affinity of Fc:FcRn by 3.5-fold, 2.2-fold, and 1.8-fold, respectively. The triple mutant has an affinity increase for FcRn at pH 6 of 12-fold relative to wild-type.

Assuming structural homology between human Fc:FcRn and rat Fc-FcRn, for which an x-ray structure was known (Burnmeister et al., Nature 372: 336-343 (1994); Burnmeister et al., Nature 372: 379-383 (1994)), Dall'Acqua et al. (Journal of Immunology. 169: 5171-5180 (2002); US2003/019031 1) pursued higher affinity improvements by phage display. They constructed four randomized libraries of Fc, each library having 4 or 5 residues completely randomized (i.e., having all possible amino acids substituted, resulting in two libraries of 20⁴ diversity, and two libraries of 20⁵ diversity) and selected for binding to murine FcRn. They reported that efforts to use human FcRn for screening the libraries were unsuccessful. Although the binding-affinity improvements identified from phage selections using murine FcRn also improved binding to human FcRn, direct phage selections using human FcRn were reportedly unsuccessful using the methods described (Dall'Acqua et al., 2002). From these libraries, they identified variants with mutations at H433, N434, and Y436 and at M252, S254, and T256. Two of their library-derived variants, H433K+N434F+Y436H and M252Y+S254T+T256E were found to have 10- to 20-fold increased affinity for both murine and human FcRn, at pH 6.0. The combination of these mutations led to a 30-fold increase in binding to murine FcRn and a 57-fold increase in binding to human FcRn. However, these variants also had increased affinity at pH 7.4, and do not have prolonged half-life in mice. This supports the conclusions that efficient IgG recycling is related to pH dependent affinity. No results were reported for these variants in primate species or in human FcRn transgenic animals.

Ward et al, U.S. Pat. No. 6,277,375, U.S. Pat. No. 6,821,505 and U.S. Pat. No. 6,165,745 describe immunoglobulin-like domains with increased half-lives and mutations at Fc positon 434. A resultant mutant N434Q actually showed reduced half-life. Israel and Simister in WO 98/23289 discuss altering residue 434 generally by addition, substitution or deletion of the residue to affect binding to FcRn but does not mention what that residue should be substituted with or what was to be added.

Also assuming structural homology to the rat Fc-FcRn complex (Burnmeister et al., 1997) to model the human Fc-FcRn interface, Hinton et al., (J. Biol. Chem. 279: 6213-6216 (2004)) identified residues T250, L314, and M428 in human IgG2 as residues that could be important for binding huFcRn. They identified mutations T250Q and M428L as having about 3-fold and 7-fold higher affinity, respectively, for human FcRn at pH 6.0, with no significant binding at pH 7.5. The combination variant T250Q+M428L was reported to have 28-fold increased binding. Similar binding was observed for rhesus monkey FcRn. Pharmacokinetic studies indicated that an IgG2 antibody with these two mutations has about a 1.9-fold longer elimination half-life (t ½ beta) in rhesus monkeys.

There is a continuing need in the art to produce antibodies, in particular therapeutic antibodies having improved or modulated effector function. One of the goals of antibody engineering is to increase the half-life of antibodies in vivo. This can be achieved by modulating the interaction of the antibody with the neonatal Fc receptor (FcRn). The present invention satisfies these and other needs.

### SUMMARY OF THE INVENTION

The present invention provides polypeptides, in particular antibodies which demonstrate higher binding affinity for FcRn and FcγRIII than polypeptides having native sequence/wild type sequence Fc region. These Fc variant polypeptides and antibodies have the advantage of being salvaged and recycled rather than degraded. Increased serum half life will be beneficial to increase exposure to antibody and reduce the frequency of administration of Fc containing polypeptides such as Abs and other antibody fusion proteins such as immunoadhesins.

The invention provides an isolated polypeptide comprising a variant IgG Fc region comprising at least an amino acid substitution at Asn 434 to Trp (N434W).

A second isolated polypeptide is one comprising a variant IgG Fc region comprising at least an amino acid substitution at Asn 434 to His (N434H).

Another isolated polypeptide provided by the invention is a polypeptide comprising a variant IgG Fc region comprising at least an amino acid substitution at Asn 434 to Tyr (N434Y) wherein the polypeptide does not further have an amino acid substitution selected from the group consisting of H433R, H433S, Y436H, Y436R, Y436T.

Yet another polypeptide is an isolated polypeptide comprising a variant IgG Fc region comprising at least an amino acid substitution at Asn 434 to Phe (N434F) wherein the polypeptide does not further have an amino acid substitution of H433K, Y436H, M252Y, S254T, or T256E.

The invention provides a polypeptide having a variant IgG Fc region wherein the variant IgG Fc region has an amino acid substitution consisting essentially of or consisting of Asn 434 to Tyr (N434Y). Also provided is a polypeptide having a variant IgG Fc wherein the variant IgG Fc has an amino acid substitution consisting essentially of or consisting of Asn 434 to Phe (N434F).

In one embodiment, the isolated polypeptide of any of the preceding embodiments is an antibody. In another embodiment, the polypeptide is an immunoadhesin.

In preferred embodiments, the IgG antibody of any of the preceding embodiments is murine or human, preferably human. Human IgG encompasses any of the human IgG isotypes of IgG1, IgG2, IgG3, IgG4. Murine IgG encompasses the isotypes of IgG1, 2a, 2b, 3. Preferably the therapeutic antibodies for human use are humanized, human or chimeric.

In the preceding polypeptides which include antibodies, the polypeptide comprising the variant Fc region binds human FcRn at pH 6.0 with higher affinity than a polypeptide comprising native sequence IgG Fc region, and binds human FcRn with weaker binding affinity at pH 7.4 or pH 7.5 than at pH 6.0. In a preferred embodiment, the binding affinity of the Fc variant polypeptide for human FcRn at pH 6.0 is at least 4-, preferably at least 7-, 9-, or even more preferably at least 20-fold higher than native sequence/native sequence Fc. The polypeptides of the preceding embodiments have a longer serum half life in primate serum, particularly human or cynomolgus monkey serum, than a polypeptide with native sequence Fc region.

Yet another aspect of the invention is an isolated polypeptide comprising a variant IgG Fc region comprising at least an amino acid substitution at Lys 334 to Leucine (K334L). In one embodiment this polypeptide binds human FcγRIII with higher affinity than a polypeptide having native sequence IgG Fc region, greater than 3-fold higher. This polypeptide also preferably exhibits increased ADCC over a polypeptide with native sequence IgG Fc region.

Also provided is an isolated polypeptide comprising a variant IgG Fc region that exhibits improvement in binding to human FcRn at pH 6, but without increased binding at pH 7.4, which comprise at least an amino acid substitution at G385H, D312H, or N315H.

In one embodiment, the isolated polypeptide of any of the preceding embodiments is an antibody. In another embodiment, the polypeptide is an immunoadhesin.

In preferred embodiments, the IgG antibody of any of the preceding embodiments is murine or human, preferably human. Human IgG encompasses any of the human IgG isotypes of IgG1, IgG2, IgG3, IgG4. Murine IgG encompasses the isotypes of IgG1, 2a, 2b, 3. Preferably the therapeutic antibodies for human use are humanized, human or chimeric.

The invention specifically provides antibodies of the preceding embodiments that bind the group of antigens consisting of CD20, Her2, BR3, TNF, VEGF, IgE, CD11a. In specific embodiments, the recombinantly produced, humanized antibodies that bind specific antigens comprise the sequences as disclosed in the SEQ ID NOs under the section subtitled antibody composition below.

In a preferred embodiment the CD20 is a primate CD20. Human and cynomolgus monkey CD20 are specific embodiments. Where the antibody binds human CD20, in more specific embodiments, the antibody will comprise a VH sequence of SEQ ID NO. 2 and a L chain that comprises the VL sequence of SEQ ID NO. 1 or the full length L chain sequence of SEQ ID NO. 26. In another embodiment, the CD20 binding antibody comprises the C2B8 VL sequence from SEQ ID NO. 24 and the VH sequence from SEQ ID NO. 25 as shown in FIG. 10. In yet another embodiments, the isolated humanized antibody that binds human CD20 will comprise the VH and VL sequences disclosed below under humanized 2H7 variants.

Where the antibody binds HER2, in more specific embodiments, the antibody will comprise V_{L} and V_{H} sequences selected from V_{L} sequence of SEQ ID NO.3 paired with V_{H} sequence of SEQ ID NO. 4; and V_{L} sequence of SEQ ID NO. 5 paired with V_{H} sequence of SEQ ID NO. 6. One specific anti-HER2 antibody comprises a variant IgG Fc region comprising at least an amino acid substitution at Asn 434 to His (N434H).

Additionally, the invention provides an isolated anti-HER2 antibody comprising V_{L} sequence of SEQ ID NO. 5, V_{H} sequence of SEQ ID NO. 6, and a variant IgG Fc region comprising at least an amino acid substitution at Asn 434 to Ala (N434A).

In preferred embodiments, the VH and VL sequences provided are joined to human IgG1 constant region, the sequence of which is shown in FIG. 4 and FIG. 5.

In one aspect, the antibodies of the preceding embodiments further comprise one or more amino acid substitutions in the Fc region that result in the antibody exhibiting one or more of the properties selected from increased FcγR binding, increased ADCC, increased CDC, decreased CDC, increased ADCC and CDC, increased ADCC but decreased CDC function, increased FcRn binding and serum half life, as compared to the antibody having native sequence Fc region.

An antibody of the preceding embodiments may further comprise one or more amino acid substitutions in the IgG Fc region at a residue position selected from the group consisting of D265A, S298A/E333A/K334A, K334L, K322A, K326A, K326W, E380A and E380A/T307A, wherein the numbering of the residues is that of the EU index as in Kabat. Wherein the polypeptide comprises an amino acid substitution of K334L, it may further comprise one or more amino acid substitutions in the IgG Fc region at a residue position selected from the group consisting of D265A, S298A/E333A, K322A, K326A, K326W, E380A and E380A/T307A.

The invention also provides a composition comprising the polypeptide or antibody of any of the preceding embodiments and a carrier, such as a pharmaceutically acceptable carrier.

Another aspect of the invention is an isolated nucleic acid encoding a polypeptide of any one of the preceding embodiments. Expression vectors encoding the polypeptides including antibodies of the invention are also provided. Also provided is a host cell comprising a nucleic acid encoding a polypeptide or antibody of the invention. Host cells that express and produce the polypeptide include CHO cell or E. coli bacterial cell. A method is also provided for producing the polypeptides, antibodies and immunoadhesins of the invention, comprising culturing a host cell comprising a nucleic acid encoding the polypeptide which host cell produces the polypeptide, and recovering the polypeptide from the cell culture.

Still another aspect of the invention is an article of manufacture comprising a container and a composition contained therein, wherein the composition comprises a polypeptide or antibody of any of the preceding embodiments. The article of manufacture can further comprise a package insert indicating that the composition can be used to treat the indication the antibody as intended for.

The invention provides a method of treating a B cell neoplasm or malignancy characterized by B cells expressing CD20, comprising administering to a patient suffering from the neoplasm or malignancy, a therapeutically effective amount of a CD20 binding antibody, in particular, a humanized CD20 binding antibody of the above embodiments. In specific embodiments, the B cell neoplasm is non-Hodgkin's lymphoma (NHL), small lymphocytic (SL) NHL, lymphocyte predominant Hodgkin's disease (LPHD), follicular center cell (FCC) lymphomas, acute lymphocytic leukemia (ALL), chronic lymphocytic leukemia (CLL) and Hairy cell leukemia.

One embodiment provides for a method of treating chronic lymphocytic leukemia, comprising administering to a patient suffering from the leukemia, a therapeutically effective amount of an antibody of comprising a variant IgG Fc of the above embodiments, which antibody binds human CD20, wherein the antibody further comprises amino acid substitution K326A or K326W.

A further aspect is a method of alleviating a B-cell regulated autoimmune disorder comprising administering to a patient suffering from the disorder, a therapeutically effective amount of a CD20 binding antibody comprising a variant IgG Fc of the above embodiments. In specific embodiments, the autoimmune disorder is selected from the group consisting of rheumatoid arthritis, juvenile rheumatoid arthritis, systemic lupus erythematosus (SLE), Wegener's disease, inflammatory bowel disease, idiopathic thrombocytopenic purpura (ITP), thrombotic throbocytopenic purpura (TTP), autoimmune thrombocytopenia, multiple sclerosis, psoriasis, IgA nephropathy, IgM polyneuropathies, myasthenia gravis, vasculitis, diabetes mellitus, Reynaud's syndrome, Sjorgen's syndrome and glomerulonephritis.

Other treatment methods provided are as follows:

A method of treating an angiogenesis related disorder is provided which comprises administering to a patient suffering from the disorder, a therapeutically effective amount of a VEGF binding antibody comprising a variant IgG Fc of the above embodiments.

A method of treating a HER2 expressing cancer, comprising administering to a patient suffering from the cancer, a therapeutically effective amount of a HER2 binding antibody that comprises a variant IgG Fc of the above embodiments.

A method of treating a LFA-1 mediated disorder comprising administering to a patient suffering from the disorder, a therapeutically effective amount of an antibody that binds human anti-CD11a comprising a variant IgG Fc of the above embodiments.

A method of treating an IgE-mediated disorder, comprising administering to a patient suffering from the disorder, a therapeutically effective amount of an antibody that binds human IgE comprising a variant IgG Fc of the above embodiments.

Yet another aspect of the invention is a method of screening for a polypeptide with higher affinity binding to FcRn at pH 6.0 and with weaker binding affinity at pH 7.4 than at pH 6.0. Preferably the polypeptide has higher affinity binding to human FcRn at pH 6.0 than a polypeptide or antibody having native sequence IgG Fc. The method comprises expressing a candidate polypeptide on phage, providing huFcRn immobilized on a solid matrix, allow phage particles to bind to the FcRn on the matrix, removing unbound phage particles by multiple rounds of washes each round with increasing stringency; and eluting the remaining bound phage at pH 7.4.

### BRIEF DESCRIPTION OF THE FIGURES

FIG. 1 is a schematic representation of a native IgG and enzymatic digestion thereof to generate various antibody fragments. Disulfide bonds are represented by S—S between CH1 and CL domains and the two CH2 domains. V is variable domain; C is constant domain; L stands for light chain and H stands for heavy chain.

FIGS. 2A and 2B show the VL (FIG. 2A; SEQ ID No.5) and VH (FIG. 2B; SEQ ID No.6) amino acid sequences of an anti-Her2 antibody (Trastuzumab).

FIGS. 3A and 3B show the sequences of the light and heavy chains of specific anti-IgE antibodies E25, E26, E27 and Hu-901.

FIG. 4 depicts alignments of native sequence human IgG Fc region sequences, humIgG1 (non-A and A allotypes; SEQ ID NOs:29 and 30, respectively), humIgG2 (SEQ ID NO:31), humIgG3 (SEQ ID NO:32) and humIgG4 (SEQ ID NO:33) with differences between the sequences marked with asterisks.

FIG. 5 depicts alignments of native sequence IgG Fc regions. Native sequence human IgG Fc region sequences, humIgG1 (non-A and A allotypes) (SEQ ID NOs:29 and 30, respectively), humIgG2 (SEQ ID NO:31), humIgG3 (SEQ ID NO:32) and humIgG4 (SEQ ID NO:33), are shown. The human IgG1 sequence is the non-A allotype, and differences between this sequence and the A allotype (at positions 356 and 358; EU numbering system) are shown below the human IgG1 sequence. Native sequence murine IgG Fc region sequences, murIgG1 (SEQ ID NO:34), murIgG2A (SEQ ID NO:35), murIgG2B (SEQ ID NO:36) and murIgG3 (SEQ ID NO:37), are also shown.

FIG. 6 depicts the role of FcRn in IgG homeostasis. The ovals within the vesicles are FcRn.

FIG. 7 shows the sequence of the human IgG1 Fc protein variant (W0437) used for phage-display. The mature protein sequence (SEQ ID NO.38) of the soluble Fc is shown; the portion of the M13 g3p used for phage display is not shown. The first residue in the mature protein sequence, Ser, corresponds to a mutation of the second Cys of the hinge region (C229), and the last residue (Leu) is the site of fusion to M13 g3p. The underlined residue corresponds to N434.

FIG. 8 shows equilibrium analysis of E. coli-produced wild-type and variant Fc binding to huFcRn at pH 6.0 by SPR (BIAcore).

FIG. 9 shows ELISA analysis of 2H7 IgG1 variants binding to human FcRn. Human IgG1 variants were produced by transient transfection in mammalian cells, and compared to humanized 4D5 (Herceptin®) for binding FcRn at pH 6.0 or pH 7.4. NeutrAvidin coat/FcRn-biotinylated/antibody/goat anti-hu-IgG-F(ab)′2-HRP association (pH 6.0) and dissociation (pH 7.4).

FIG. 10 shows the C2B8 light (SEQ ID NO.24) and heavy chain (SEQ ID NO.25) sequences. The constant and Fc regions are boxed and the variable regions are outside of the box.

FIG. 11 shows binding affinity of 2H7 variants to human FcγRIII (V158) in an ELISA.

FIG. 12 shows the serum concentration-time profile of PRO145234, PRO145181, and PRO145182 following a single IV Dose of 20 mg/kg in Cynomolgus monkeys.

FIG. 13 shows the binding of Herceptin and hu4D5(N434H) to human FcRn at pH 6.0 and pH 7.4, as assayed by ELISA.

### DETAILED DESCRIPTION OF THE PREFERRED EMBODIMENTS

An important component of the homeostasis of IgG is the recycling pathway mediated by the pH dependent interaction of the Fc region with the cell-surface neonatal receptor, FcRn. An important goal for the field of antibody engineering has been to identify mutations in the Fc that increase the affinity of the Fc-FcRn complex at pH 6.0, while retaining low affinity at pH 7.4 (Ghetie et al., 1997). Furthermore, it is highly desirable to minimize the number of mutations introduced to the Fc to avoid potential anti-drug immune responses in patients treated with therapeutic antibodies that include mutations to the highly conserved constant domains. In the present invention we identified single amino acid mutations (N434W, N434Y, and N434F; the numbering system used here for the IgG Fc region is the EU notation as described in Kabat, Sequences of Proteins of Immunological Interest (1991)) that increase the affinity of Fc for human FcRn, the N434W mutant increased Fc binding affinity by about 170-fold at pH 6.0 and retain low affinity for huFcRn at pH 7.4, through the use of phage-display and a novel method for constructing libraries of randomized amino acids.

Methods of measuring binding to FcRn are known (see, e.g., Ghetie 1997, Hinton 2004) as well as described in the examples. Binding to human FcRn in vivo and serum half life of human FcRn high affinity binding polypeptides can be assayed, e.g, in transgenic mice or transfected human cell lines expressing human FcRn, or in primates administered with the Fc variant polypeptides. In separate embodiments, the polypeptide and specifically the antibody of the invention having a variant IgG Fc exhibits increased binding affinity for human FcRn over a polypeptide having wild-type IgG Fc, by at least 7 fold, at least 9 fold, more preferably at least 20 fold, preferably at least 40 fold, even more preferably at least 70 to 100 fold. In a specific embodiment, the binding affinity for human FcRn is increased about 70 fold.

The invention also provides an isolated polypeptide comprising a variant IgG Fc region comprising at least an amino acid substitution at Lys 334 to Leucine (K334L). This polypeptide binds human FcγRIII with higher affinity than native sequence IgG Fc, greater than 3-fold higher. These polypeptides preferably exhibit increased ADCC in the presence of human effector cells over a polypeptide with native sequence IgG Fc. Where the antibody is a CD20 binding antibody, ADCC activity can be tested in transgenic mice expressing human CD20 plus CD16 (hCD20+/hCD16+Tg mice). Assays for ADCC have been described, see, e.g., Presta U.S. Pat. No. 6,737,056.

For binding affinity to FcRn, in one embodiment, the EC50 or apparent Kd (at pH 6.0) of the polypeptide is <=100 nM, more preferably <=10 nM. For increased binding affinity to FcγRIII (F158; i.e. low-affinity isotype), in one embodiment the EC50 or apparent Kd <=10 nM, and for FcgRIII (V158; high-affinity) the EC50 or apparent Kd <=3 nM.

Throughout the present specification and claims, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

A “parent polypeptide” is a polypeptide comprising an amino acid sequence which lacks one or more of the Fc region modifications disclosed herein and which differs in effector function compared to a polypeptide variant as herein disclosed. The parent polypeptide may comprise a native sequence Fc region or an Fc region with pre-existing amino acid sequence modifications (such as additions, deletions and/or substitutions).

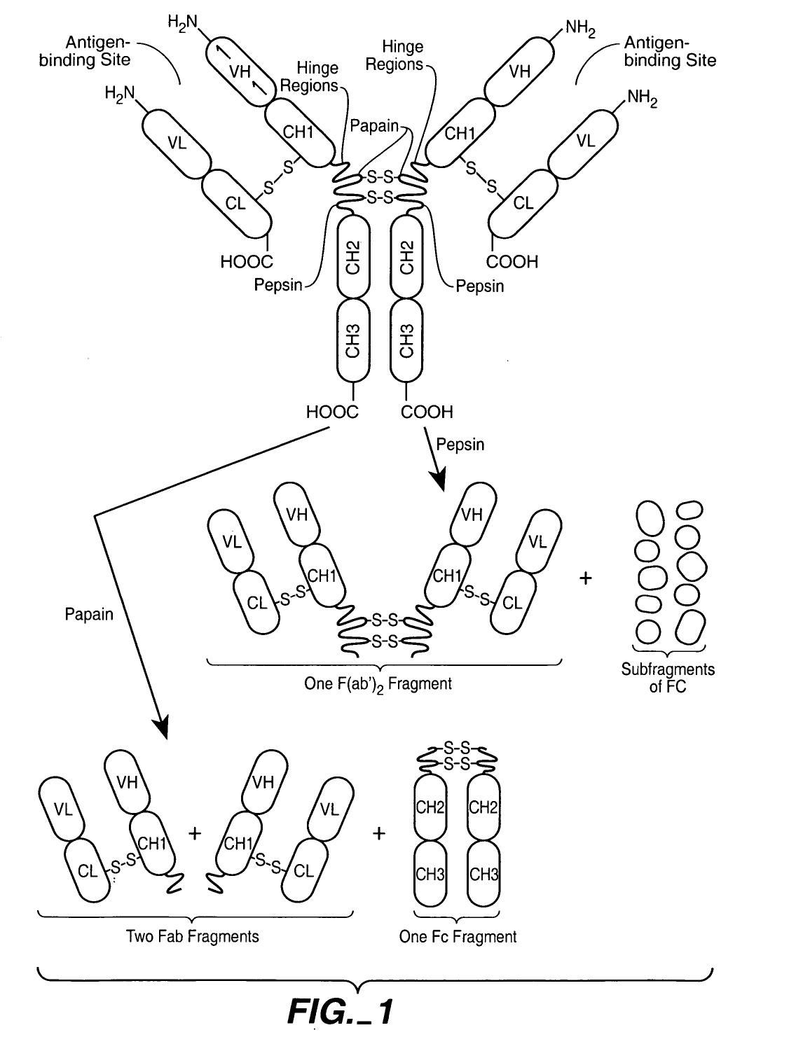

The term “Fc region” is used to define a C-terminal region of an immunoglobulin heavy chain, e.g., as shown in FIG. 1. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The Fc region of an immunoglobulin generally comprises two constant domains, CH2 and CH3, as shown, for example, in FIG. 1. The last residue, lysine, in the heavy chain of IgG1 can but does not have to be present as the terminal residue in the Fc in the mature protein.

The “CH2 domain” of a human IgG Fc region (also referred to as “Cγ2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec. Immunol. 22:161-206 (1985).

The “CH3 domain” comprises the stretch of residues C-terminal to a CH2 domain in an Fc region (i.e. from about amino acid residue 341 to about amino acid residue 447 of an IgG)

A “functional Fc region” possesses an “effector function” of a native sequence Fc region. Exemplary “effector functions” include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays as herein disclosed, for example.

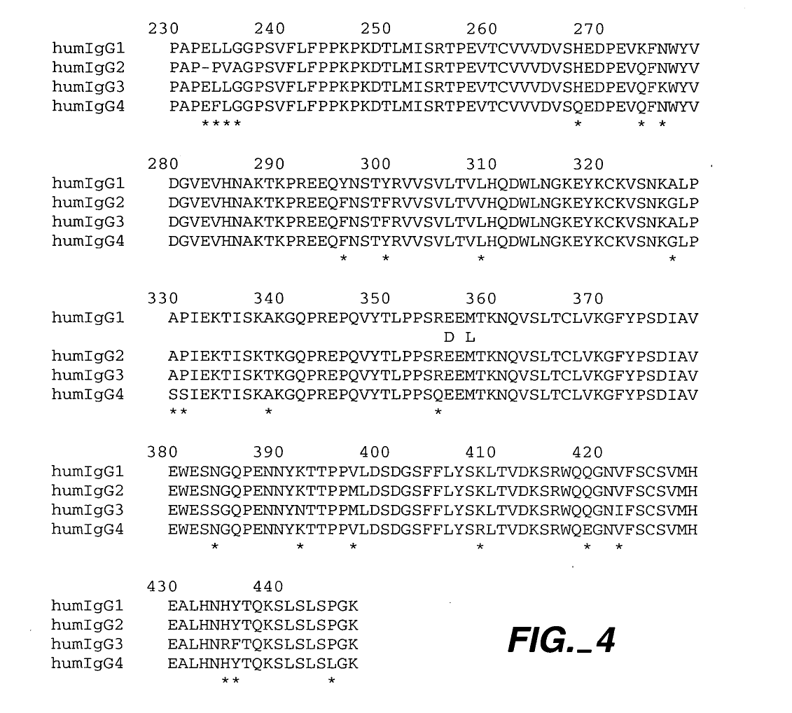

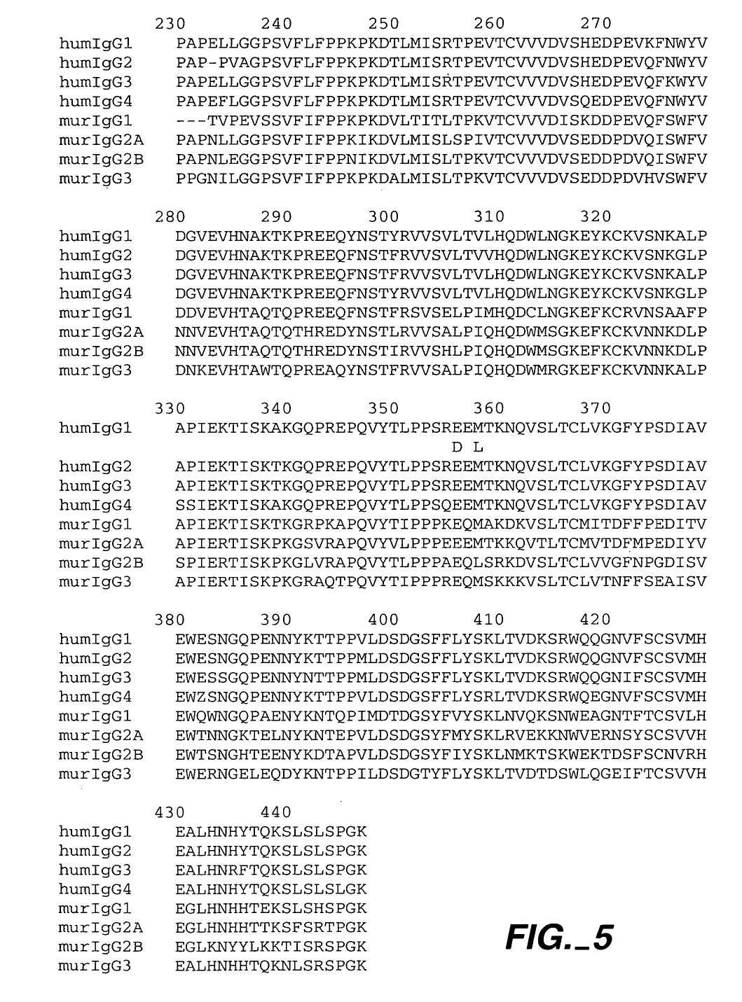

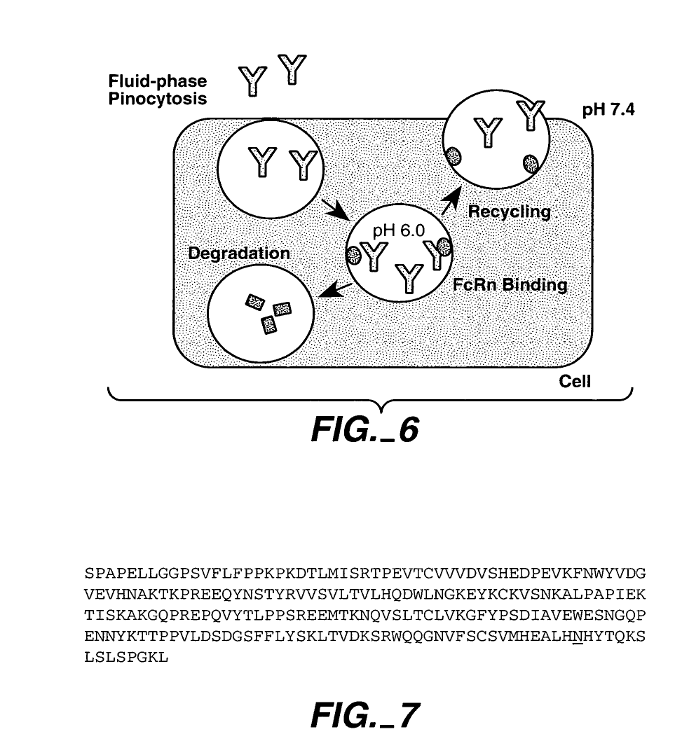

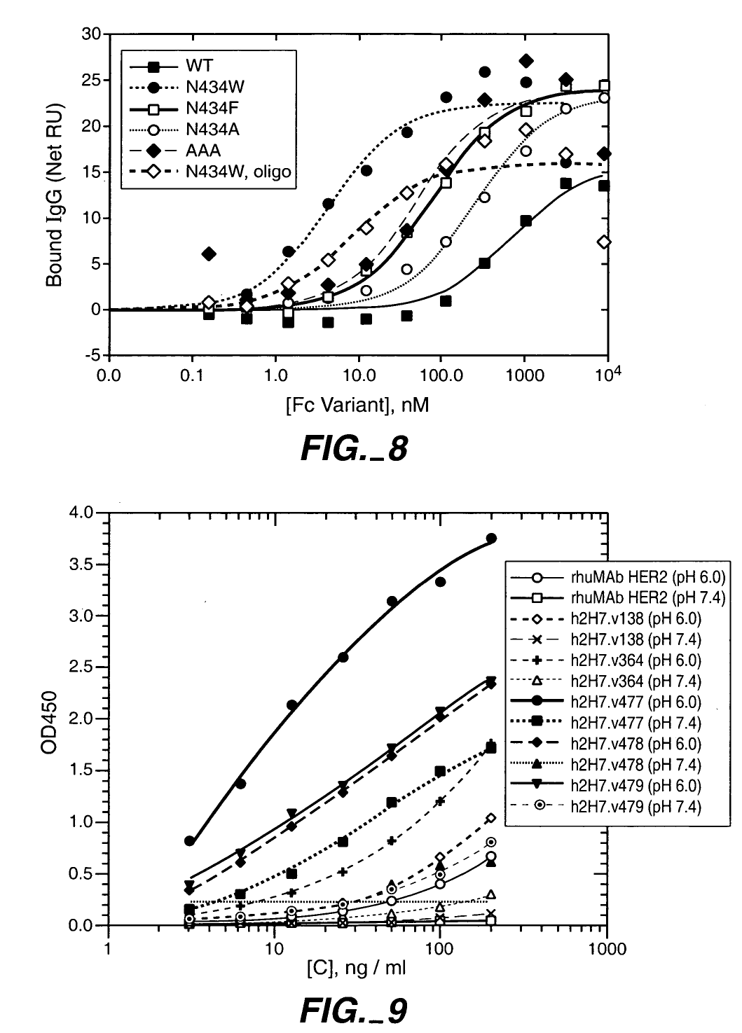

A “native sequence Fc region” comprises an amino acid sequence identical to the amino acid sequence of an Fc region found in nature. Native sequence human Fc regions are shown in FIGS. 4 and 5 and include a native sequence human IgG1 Fc region (non-A and A allotypes); native sequence human IgG2 Fc region; native sequence human IgG3 Fc region; and native sequence human IgG4 Fc region as well as naturally occurring variants thereof. Native sequence murine Fc regions are shown in FIG. 5.

A “variant Fc region” comprises an amino acid sequence which differs from that of a native sequence Fc region by virtue of at least one “amino acid modification” as herein defined. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Homology” is defined as the percentage of residues in the amino acid sequence variant that are identical after aligning the sequences and introducing gaps, if necessary, to achieve the maximum percent homology. Methods and computer programs for the alignment are well known in the art. One such computer program is “Align 2”, authored by Genentech, Inc., which was filed with user documentation in the United States Copyright Office, Washington, D.C. 20559, on Dec. 10, 1991.

The term “Fc region-containing polypeptide” refers to a polypeptide, such as an antibody or immunoadhesin (see definitions below), which comprises an Fc region.

The terms “Fc receptor” or “FcR” are used to describe a receptor that binds to the Fc region of an IgG antibody. The preferred FcR is a native sequence human FcR. In one embodiment, the FcR is a FcγR which includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daëron, Annu. Rev. Immunol. 15:203-234 (1997)). The term includes allotypes, such as FcγRIIIA allotypes: FcγRIIIA-Phe158, FcγRIIIA-Val158, FcγRIIA-R131 and/or FcγRIIA-H131. FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Antibody-dependent cell-mediated cytotoxicity” or “ADCC” refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g. from blood or PBMCs as described herein.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g. as described in Gazzano-Santoro et al., J. Immunol. Methods 202:163 (1996), may be performed.

A polypeptide with a variant IgG Fc with “altered” FcR binding affinity or ADCC activity is one which has either enhanced or diminished FcR binding activity (FcγR or FcRn) and/or ADCC activity compared to a parent polypeptide or to a polypeptide comprising a native sequence Fc region. The variant Fc which “exhibits increased binding” to an FcR binds at least one FcR with better affinity than the parent polypeptide. The improvement in binding compared to a parent polypeptide may be about 3 fold, preferably about 5, 10, 25, 50, 60, 100, 150, 200, up to 500 fold, or about 25% to 1000% improvement in binding. The polypeptide variant which “exhibits decreased binding” to an FcR, binds at least one FcR with worse affinity than a parent polypeptide. The decrease in binding compared to a parent polypeptide may be about 40% or more decrease in binding. Such Fc variants which display decreased binding to an FcR may possess little or no appreciable binding to an FcR, e.g., 0-20% binding to the FcR compared to a native sequence IgG Fc region, e.g. as determined in the Examples herein.

The polypeptide having a variant Fc which binds an FcR with “better affinity” of “higher affinity” than a polypeptide or parent polypeptide having wild type or native sequence IgG Fc is one which binds any one or more of the above identified FcRs with substantially better binding affinity than the parent polypeptide with native sequence Fc, when the amounts of polypeptide with variant Fc and parent polypeptide in the binding assay are essentially the same. For example, the variant Fc polypeptide with improved FcR binding affinity may display from about 2 fold to about 300 fold, e.g. from about 3 fold to about 170 fold improvement in FcR binding affinity compared to the parent polypeptide, where FcR binding affinity is determined, for example, as disclosed in the Examples herein.

The polypeptide comprising a variant Fc region which “exhibits increased ADCC” or mediates antibody-dependent cell-mediated cytotoxicity (ADCC) in the presence of human effector cells more effectively than a polypeptide having wild type IgG Fc is one which in vitro or in vivo is substantially more effective at mediating ADCC, when the amounts of polypeptide with variant Fc region and the polypeptide with wild type Fc region sed in the assay are essentially the same. Generally, such variants will be identified using the in vitro ADCC assay as herein disclosed, but other assays or methods for determining ADCC activity, e.g. in an animal model etc, are contemplated. The preferred variant is from about 5 fold to about 100 fold, e.g. from about 25 to about 50 fold, more effective at mediating ADCC than the wild type Fc.

An “amino acid modification” refers to a change in the amino acid sequence of a predetermined amino acid sequence. Exemplary modifications include an amino acid substitution, insertion and/or deletion. The preferred amino acid modification herein is a substitution.

An “amino acid modification at” a specified position, e.g. of the Fc region, refers to the substitution or deletion of the specified residue, or the insertion of at least one amino acid residue adjacent the specified residue. By insertion “adjacent” a specified residue is meant insertion within one to two residues thereof. The insertion may be N-terminal or C-terminal to the specified residue.

An “amino acid substitution” refers to the replacement of at least one existing amino acid residue in a predetermined amino acid sequence with another different “replacement” amino acid residue. The replacement residue or residues may be “naturally occurring amino acid residues” (i.e. encoded by the genetic code) and selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (lie): leucine (Leu); lysine (Lys); methionine (Met); phenylalanine (Phe); proline (Pro); serine (Ser); threonine (Thr); tryptophan (Trp); tyrosine (Tyr); and valine (Val). Preferably, the replacement residue is not cysteine. Substitution with one or more non-naturally occurring amino acid residues is also encompassed by the definition of an amino acid substitution herein. A “non-naturally occurring amino acid residue” refers to a residue, other than those naturally occurring amino acid residues listed above, which is able to covalently bind adjacent amino acid residues(s) in a polypeptide chain. Examples of non-naturally occurring amino acid residues include norleucine, ornithine, norvaline, homoserine and other amino acid residue analogues such as those described in Ellman et al. Meth. Enzym. 202:301-336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA.

The term “conservative” amino acid substitution as used within this invention is meant to refer to amino acid substitutions which substitute functionally equivalent amino acids. Conservative amino acid changes result in silent changes in the amino acid sequence of the resulting peptide. For example, one or more amino acids of a similar polarity act as functional equivalents and result in a silent alteration within the amino acid sequence of the peptide. In general, substitutions within a group may be considered conservative with respect to structure and function. However, the skilled artisan will recognize that the role of a particular residue is determined by its context within the three-dimensional structure of the molecule in which it occurs. For example, Cys residues may occur in the oxidized (disulfide) form, which is less polar than the reduced (thiol) form. The long aliphatic portion of the Arg side chain may constitute a critical feature of its structural or functional role, and this may be best conserved by substitution of a nonpolar, rather than another basic residue. Also, it will be recognized that side chains containing aromatic groups (Trp, Tyr, and Phe) can participate in ionic-aromatic or “cation-pi” interactions. In these cases, substitution of one of these side chains with a member of the acidic or uncharged polar group may be conservative with respect to structure and function. Residues such as Pro, Gly, and Cys (disulfide form) can have direct effects on the main chain conformation, and often may not be substituted without structural distortions.

An “amino acid insertion” refers to the incorporation of at least one amino acid into a predetermined amino acid sequence. While the insertion will usually consist of the insertion of one or two amino acid residues, the present application contemplates larger “peptide insertions”, e.g. insertion of about three to about five or even up to about ten amino acid residues. The inserted residue(s) may be naturally occurring or non-naturally occurring as disclosed above.

An “amino acid deletion” refers to the removal of at least one amino acid residue from a predetermined amino acid sequence.

Amino acids may be grouped according to similarities in the properties of their side chains (in A. L. Lehninger, in Biochemistry, second ed., pp. 73-75, Worth Publishers, New York (1975)):
(1) non-polar: Ala (A), Val (V), Leu (L), Ile (I), Pro (P), Phe (F), Trp (W), Met (M)
(2) uncharged polar: Gly (G), Ser (S), Thr (T), Cys (C), Tyr (Y), Asn (N), Gln (Q)
(3) acidic: Asp (D), Glu (E)
(4) basic: Lys (K), Arg (R), His(H)

Alternatively, naturally occurring residues may be divided into groups based on common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

“Hinge region” is generally defined as stretching from Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

The “lower hinge region” of an Fc region is normally defined as the stretch of residues immediately C-terminal to the hinge region, i.e. residues 233 to 239 of the Fc region. Prior to the present invention, FcγR binding was generally attributed to amino acid residues in the lower hinge region of an IgG Fc region.

“C1q” is a polypeptide that includes a binding site for the Fc region of an immunoglobulin. C1q together with two serine proteases, C1r and C1s, forms the complex C1, the first component of the complement dependent cytotoxicity (CDC) pathway. Human C1q can be purchased commercially from, e.g. Quidel, San Diego, Calif.

The term “binding domain” refers to the region of a polypeptide that binds to another molecule. In the case of an FcR, the binding domain can comprise a portion of a polypeptide chain thereof (e.g. the α chain thereof) which is responsible for binding an Fc region. One useful binding domain is the extracellular domain of an FcR α chain.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

“Functional fragments”, of the antibodies of the invention comprise a portion of an intact antibody, generally including the antigen binding or variable region of the intact antibody or the Fc region of an antibody which retains FcR binding capability. Examples of antibody fragments include linear antibodies; single-chain antibody molecules; and multispecific antibodies formed from antibody fragments.

The term “monoclonal antibody” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Methods of making chimeric antibodies are known in the art.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences. These modifications are made to further refine and maximize antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence although the FR regions may include one or more amino acid substitutions that improve binding affinity. The number of these amino acid substitutions in the FR are typically no more than 6 in the H chain, and in the L chain, no more than 3. The humanized antibody optimally also will comprise at least a portion of an immunoglobulin constant region (Fc), typically that of a human immunoglobulin. For further details, see Jones et al., Nature, 321:522-525 (1986); Reichmann et al., Nature, 332:323-329 (1988); and Presta, Curr. Op. Struct. Biol., 2:593-596 (1992). The humanized antibody includes a PRIMATIZED® antibody wherein the antigen-binding region of the antibody is derived from an antibody produced by, e.g., immunizing macaque monkeys with the antigen of interest. Methods of making humanized antibodies are known in the art.

Human antibodies can also be produced using various techniques known in the art, including phage-display libraries. Hoogenboom and Winter, J. Mol. Biol., 227:381 (1991); Marks et al., J. Mol. Biol., 222:581 (1991). The techniques of Cole et al. and Boerner et al. are also available for the preparation of human monoclonal antibodies. Cole et al., Monoclonal Antibodies and Cancer Therapy, Alan R. Liss, p. 77 (1985); Boerner et al., J. Immunol., 147(1):86-95 (1991).

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin can be obtained from any immunoglobulin, such as IgG-1, IgG-2, IgG-3, or IgG-4 subtypes, IgA (including IgA-1 and IgA-2), IgE, IgD or IgM. For example, useful immunoadhesins according to this invention are polypeptides that comprise the BLyS binding portions of a BLyS receptor without the transmembrane or cytoplasmic sequences of the BLyS receptor. In one embodiment, the extracellular domain of BR3, TACI or BCMA is fused to a constant domain of an immunoglobulin sequence.

A “fusion protein” and a “fusion polypeptide” refer to a polypeptide having two portions covalently linked together, where each of the portions is a polypeptide having a different property. The property may be a biological property, such as activity in vitro or in vivo. The property may also be a simple chemical or physical property, such as binding to a target molecule, catalysis of a reaction, etc. The two portions may be linked directly by a single peptide bond or through a peptide linker containing one or more amino acid residues. Generally, the two portions and the linker will be in reading frame with each other.

An “isolated” polypeptide or antibody is one which has been identified and separated and/or recovered from a component of its natural environment. Contaminant components of its natural environment are materials which would interfere with diagnostic or therapeutic uses for the polypeptide or antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The biological activity of the CD20 binding and humanized CD20 binding antibodies of the invention will include at least binding of the antibody to human CD20, more preferably binding to human and other primate CD20 (including cynomolgus monkey, rhesus monkey, chimpanzees). The antibodies would bind CD20 with a K_{d} value of no higher than 1×10⁻⁸, preferably a K_{d} value no higher than about 1×10⁻⁹, and be able to kill or deplete B cells in vivo, preferably by at least 20% when compared to the appropriate negative control which is not treated with such an antibody. B cell depletion can be a result of one or more of ADCC, CDC, or other mechanism. In some embodiments of disease treatment herein, specific effector functions or mechanisms may be desired over others and certain variants of the humanized 2H7 are preferred to achieve those biological functions, such as ADCC.

“Treating” or “treatment” or “alleviation” refers to therapeutic treatment wherein the object is to lessen or slow down the targeted pathologic condition or disorder. A subject is successfully “treated” for example, a CD20 positive cancer or an autoimmune disease if, after receiving a therapeutic amount of a CD20 binding antibody of the invention according to the methods of the present invention, the subject shows observable and/or measurable reduction in or absence of one or more signs and symptoms of the particular disease. For example, for cancer, reduction in the number of cancer cells or absence of the cancer cells; reduction in the tumor size; inhibition (i.e., slow to some extent and preferably stop) of tumor metastasis; inhibition, to some extent, of tumor growth; increase in length of remission, and/or relief to some extent, one or more of the symptoms associated with the specific cancer; reduced morbidity and mortality, and improvement in quality of life issues. Reduction of the signs or symptoms of a disease may also be felt by the patient. Treatment can achieve a complete response, defined as disappearance of all signs of cancer, or a partial response, wherein the size of the tumor is decreased, preferably by more than 50 percent, more preferably by 75%. A patient is also considered treated if the patient experiences stable disease. In a preferred embodiment, the cancer patients are still progression-free in the cancer after one year, preferably after 15 months. These parameters for assessing successful treatment and improvement in the disease are readily measurable by routine procedures familiar to a physician of appropriate skill in the art.

A “therapeutically effective amount” refers to an amount of an antibody or a drug effective to “treat” a disease or disorder in a subject. In the case of cancer, the therapeutically effective amount of the drug may reduce the number of cancer cells; reduce the tumor size; inhibit (ie., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See preceding definition of “treating”. To the extent the drug may prevent growth and/or kill existing cancer cells, it may be cytostatic and/or cytotoxic.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below.

A “growth inhibitory agent” when used herein refers to a compound or composition which inhibits growth of a cell, especially a CD20 expressing cancer cell, either in vitro or in vivo. Thus, the growth inhibitory agent may be one which significantly reduces the percentage of PSCA expressing cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (W B Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

A “chemotherapeutic agent” is a chemical compound useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “mammal” refers to any animal classified as a mammal, including humans, domestic and farm animals, and zoo, sports, or pet animals, such as dogs, horses, cats, cows, etc. Preferably, the mammal herein is human.

Compositions

In specific embodiments, the antibodies will comprise the V domain sequences or full length sequences shown below but will have the Fc mutations of the present invention that improve one or more of the Fc effector functions.

The polypeptides and antibodies of the present invention may further comprise other amino acid substitutions that, e.g., improve or reduce other Fc function or further improve the same Fc function, increase antigen binding affinity, increase stability, alter glycosylation, or include allotypic variants. The antibodies may further comprise one or more amino acid substitutions in the Fc region that result in the antibody exhibiting one or more of the properties selected from increased FcγR binding, increased ADCC, increased CDC, decreased CDC, increased ADCC and CDC function, increased ADCC but decreased CDC function (e.g., to minimize infusion reaction), increased FcRn binding and serum half life, as compared to the polypeptide and antibodies that have wild type Fc. These activities can be measured by the methods described herein.

For additional amino acid alterations that improve Fc function, see U.S. Pat. No. 6,737,056, incorporated herein by reference. Any of the antibodies of the present invention may further comprise at least one amino acid substitution in the Fc region that decreases CDC activity, for example, comprising at least the substitution K322A. See U.S. Pat. No. 6,528,624B1 (Idusogie et al.). Mutations that improve ADCC and CDC include S298A/E333A/K334A also referred to herein as the triple Ala mutant. K334L increases binding to CD16. K322A results in reduced CDC activity; K326A or K326W enhances CDC activity D265A results in reduced ADCC activity. Glycosylation variants that increase ADCC function are described in WO 03/035835 incorporated herein by reference. Stability variants are variants that show improved stability with respect to e.g., oxidation, deamidation.

A recombinant humanized version of the murine HER2 antibody 4D5 (huMAb4D5-8, rhuMAb HER2, Trastuzumab or HERCEPTIN®; U.S. Pat. No. 5,821,337) is clinically active in patients with HER2-overexpressing metastatic breast cancers that have received extensive prior anti-cancer therapy (Baselga et al., J. Clin. Oncol. 14:737-744 (1996)). Trastuzumab received marketing approval from the Food and Drug Administration Sep. 25, 1998 for the treatment of patients with metastatic breast cancer whose tumors overexpress the HER2 protein.

Other HER2 antibodies with various properties have been described in Tagliabue et al. Int. J. Cancer 47:933-937 (1991); McKenzie et al. Oncogene 4:543-548 (1989); Maier et al. Cancer Res. 51:5361-5369 (1991); Bacus et al. Molecular Carcinogenesis 3:350-362 (1990); Stancovski et al. PNAS (USA) 88:8691-8695 (1991); Bacus et al. Cancer Research 52:2580-2589 (1992); Xu et al. Int. J. Cancer 53:401-408 (1993); WO94/00136; Kasprzyk et al. Cancer Research 52:2771-2776 (1992); Hancock et al. Cancer Res. 51:4575-4580 (1991); Shawver et al. Cancer Res. 54:1367-1373 (1994); Arteaga et al. Cancer Res. 54:3758-3765 (1994); Harwerth et al. J. Biol. Chem. 267:15160-15167 (1992); U.S. Pat. No. 5,783,186; and Klapper et al. Oncogene 14:2099-2109 (1997).

In one embodiment, the anti-HER2 antibody comprises the following VL and VH domain sequences (the CDRs are indicated in bold): humanized 2C4 version 574 antibody VL (SEQ ID NO:3)

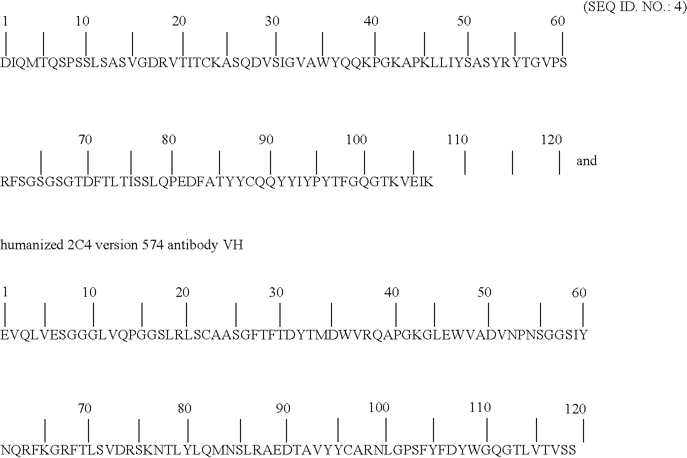

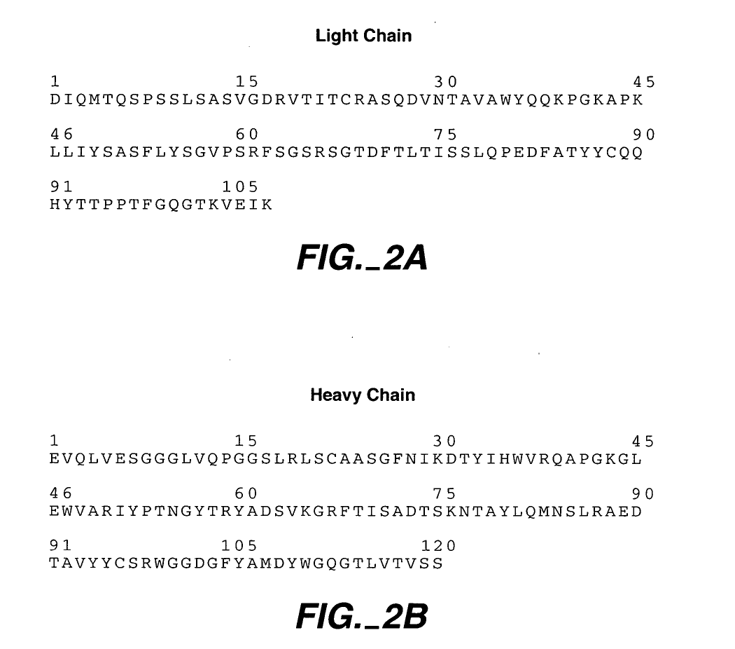

In another embodiment, the anti-HER2 antibody comprises the VL (SEQ ID NO.5) and VH (SEQ ID NO.6) domain sequences of Trastuzumab as shown in FIG. 2A and FIG. 2B.

In specific embodiments, the anti-VEGF antibodies of the invention comprise the following sequences:

In one embodiment, the anti-VEGF antibody comprises VL sequence of: (SEQ ID NO:7)

| | |
|---|---|
| In one embodiment, the anti-VEGF antibody comprises | |
| VL sequence of: | |
| DIQMTQTTSS LSASLGDRVI ISCSASQDIS NYLWWYQQKP DGTVKVLIYF | (SEQ ID NO: 7) |
| TSSLHSGVPS RFSGSGSGTD YSLTISNLEP EDIATYYCQQ YSTVPWTFGG | |
| GTKLEIKR; | |
| and | |
| VH sequence of: | |
| EIQLVQSGPE LKQPGETVRI SCKASGYTFT NYGMNWVKQA PGKGLKWMGW | (SEQ ID NO:8) |
| INTYTGEPTY AADFKRRFTF SLETSASTAY LQISNLKNDD TATYFCAKYP | |
| HYYGSSHWYF DVWGAGTTVT VSS; | |
| In another embodiment, the anti-VEGF antibody comprises | |
| VL sequence of: | |
| DIQMTQSPSS LSASVGDRVT ITCSASQDIS NYLNWYQQKP GKAPKVLIYF | (SEQ ID NO:9) |
| TSSLHSGVPS RFSGSGSGTD FTLTISSLQP EDFATYYCQQ YSTVPWTFGQ | |
| GTKVEIKR; | |
| and | |
| VH sequence of: | |
| EVQLVESGGG LVQPGGSLRL SCAASGYTFT NYGMNWVRQA PGKGLEWVGW | (SEQ ID NO:10) |
| INTYTGEPTY AADFKRRFTF SLDTSKSTAY LQMNSLRAED TAVYYCAKYP | |
| HYYGSSHWYF DVWGQGTLVT VSS. | |
| In a third embodiment, the anti-VEGF antibody comprises | |
| VL sequence of: | |
| DIQLTQSPSS LSASVGDRVT ITCSASQDIS NYLNWYQQKP GKAPKVLIYF | (SEQ ID NO:11) |
| TSSLHSGVPS RFSGSGSGTD FTLTISSLQP EDFATYYCQQ YSTVPWTFGQ | |
| GTKVEIKR; | |
| and | |
| VH sequence of: | |
| EVQLVESGGG LVQPGGSLRL SCAASGYDFT HYGMNWVRQA PGKGLEWVGW | (SEQ ID NO:12) |
| INTYTGEPTY AADFKRRFTF SLDTSKSTAY LQMNSLRAED TAVYYCAKYP | |
| YYYGTSHWYF DVWGQGTLVT VSS | |

The humanized anti-CD11a antibody efalizumab or Raptiva® (U.S. Pat. No. 6,037,454) received marketing approval from the Food and Drug Administration on Oct. 27, 2003 for the treatment for the treatment of psoriasis. One embodiment provides for an anti-human CD11a antibody comprising the Fc mutations of the present invention that improve one or more of the Fc effector functions, the antibody comprising the VL and VH sequences of HuMHM24 below:

Variable Light (SEQ ID NO:13)

| HuMHM24 | DIQMTQSPSSLSASVGDRVTTTCRASKTISKYLAWYQQKPGKAPKLLIY |
|---|---|
| | 1 10 20 30 40 |
| HuMHM24 | SGSTLQSGVPSRFSGSGSGTDFTLTISSLQPEDFATYYCQQHNEYPLTFGQ |
| | 60 70 80 90 100 |
| HuMHM24 | GTKVEIKR |
| Variable Heavy | |
| (SEQ ID NO: 14) | |
| HuMHM24 | EVQLVESGGGLVQPGGSLRLSCAASGYSFTGHWMNWVRQAPGKGLEWV |
| | 1 10 20 30 40 |
| HuMHM24 | GMIHPSDSETRYNQKFKDRFTISVDKSKNTLYLQMNSLRAEDTAVYYCAR |
| | 50 a 60 70 80 abc 90 |
| HuMHM24 | GIYFYGTTYFDYWGQGTLVTVSS |
| | 100 110 |

The anti-human CD11a antibody may comprise the VH of SEQ ID NO:14 and the full length L chain of HuMHM24 having the sequence of:

(SEQ ID NO: 15)

DIQMTQSPSSLSASVGDRVTTTCRASKTISKYLAWYQQKPGKAPKLLIYS
GSTLQSGVPSRFSGSGSGTDFTLTISSLQPEDFATYYCQQHNEYPLTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

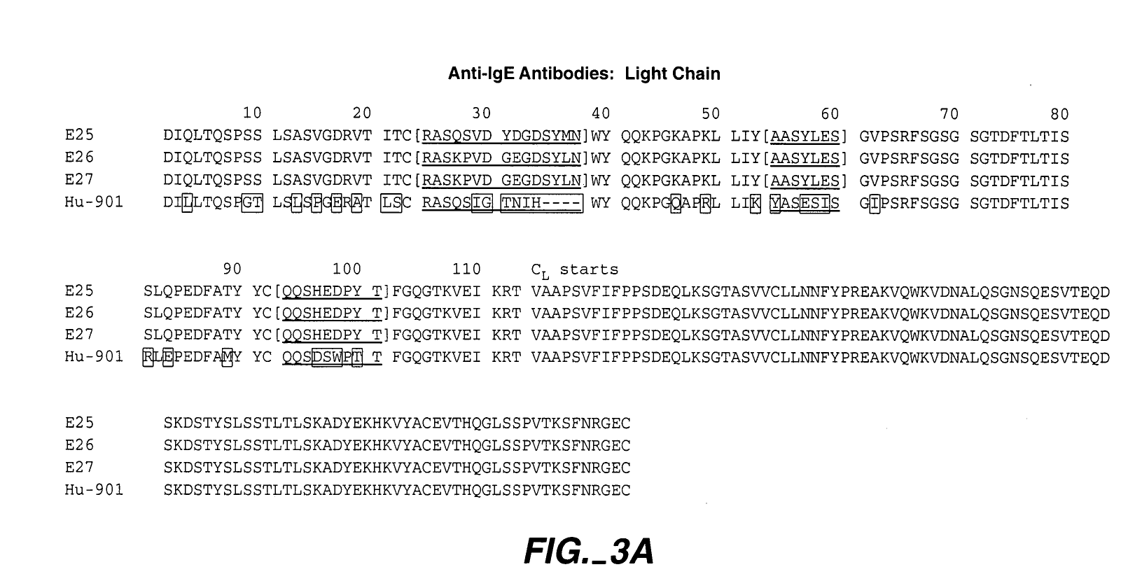

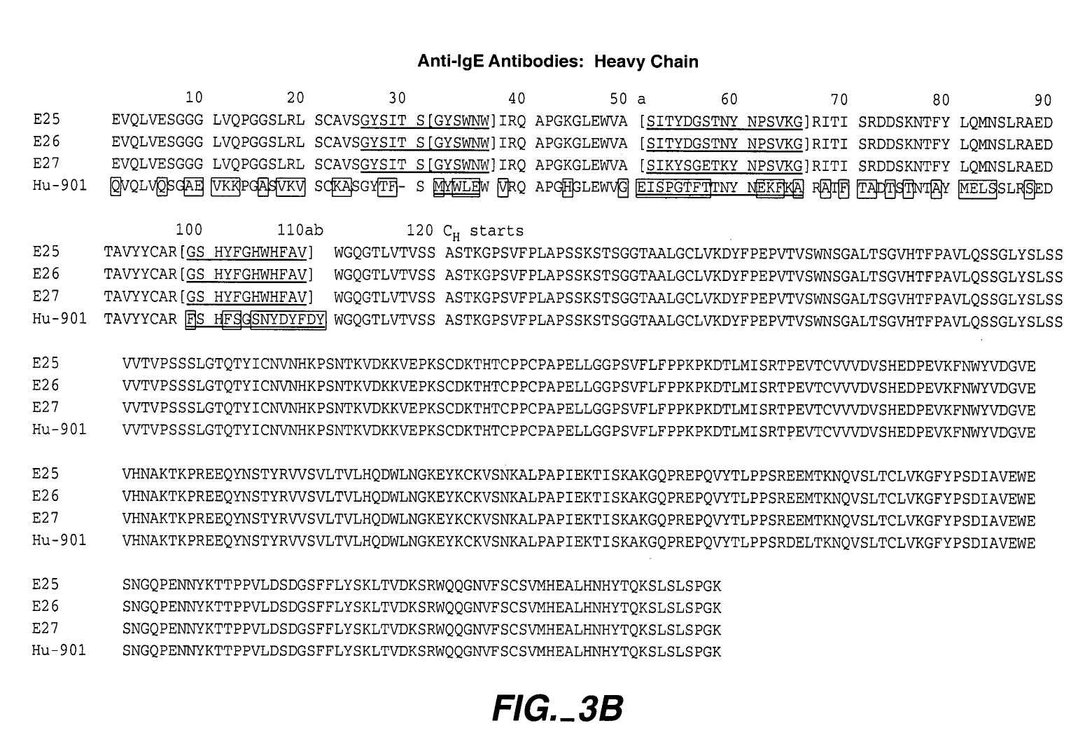

In specific embodiments, the anti-IgE antibodies having the Fc mutations of the present invention that improve one or more of the Fc effector functions comprise at least the V region sequences of the anti-IgE antibodies E25, E26, E27 and Hu-901, the L and H chain sequences of which are shown in FIGS. 3A and 3B. The light chain sequences are as follows: E25L chain (SEQ ID NO.16); E26 L chain (SEQ ID NO.17); E27 L chain (SEQ ID NO.18); and Hu-901 L chain (SEQ ID NO.19). The heavy chains sequences are as follows: E25 H chain (SEQ ID NO.20 ); E26 H chain (SEQ ID NO.21); E27 H chain (SEQ ID NO.22); and Hu-901 H chain (SEQ ID NO.23). For the anti-IgE antibodies shown in FIGS. 3A and 3B, the VL ends at VEIK (residue 111 in FIG. 3A) and the VH ends at VTVSS [around residue#121 in FIG. 3B). The VL sequences of E25, E26, E27 and Hu-901 antibodies are as SEQ ID NO.47, SEQ ID NO.49, SEQ ID NO.51 and SEQ ID NO.53, respectively. The VH sequences of E25, E26, E27 and Hu-901 antibodies are as SEQ ID NO.48, SEQ ID NO.50, SEQ ID NO.52 and SEQ ID NO.54, respectively. In another embodiment, the anti-IgE antibodies having the Fc mutations of the present invention will comprise a L chain selected from any one of the antibodies whose sequences are shown in FIG. 3A: E25 L chain (SEQ ID NO.16); E26 L chain (SEQ ID NO.17); E27 L chain (SEQ ID NO.18); and Hu-901 L chain (SEQ ID NO.19).

Examples of antibodies which bind the CD20 antigen include: “C2B8” which is now called “Rituximab” (“RITUXAN®”) (U.S. Pat. No. 5,736,137, expressly incorporated herein by reference); the yttrium-[90]-labeled 2B8 murine antibody designated “Y2B8” or “Ibritumomab Tiuxetan” ZEVALIN® (U.S. Pat. No. 5,736,137, expressly incorporated herein by reference); murine IgG2a “B1,” also called “Tositumomab,” optionally labeled with ¹³¹I to generate the “131I-B1” antibody (iodine I131 tositumomab, BEXXAR™) (U.S. Pat. No. 5,595,721, expressly incorporated herein by reference); murine monoclonal antibody “1F5” (Press et al. Blood 69(2):584-591 (1987) and variants thereof including “framework patched” or humanized IF5 (WO03/002607, Leung, S.); ATCC deposit HB-96450); murine 2H7 and chimeric 2H7 antibody (Clark et al. PNAS 82: 1766-1770 (1985); U.S. Pat. No. 5,500,362, expressly incorporated herein by reference); humanized 2H7; huMax-CD20 (WO 04/035607, Genmab, Denmark); AME-133 (Applied Molecular Evolution); A20 antibody or variants thereof such as chimeric or humanized A20 antibody (cA20, hA20, respectively) (US 2003/0219433, Immunomedics); and monoclonal antibodies L27, G28-2, 93-IB3, B-Cl or NU-B2 available from the International Leukocyte Typing Workshop (Valentine et al., In: Leukocyte Typing III (McMichael, Ed., p. 440, Oxford University Press (1987)).

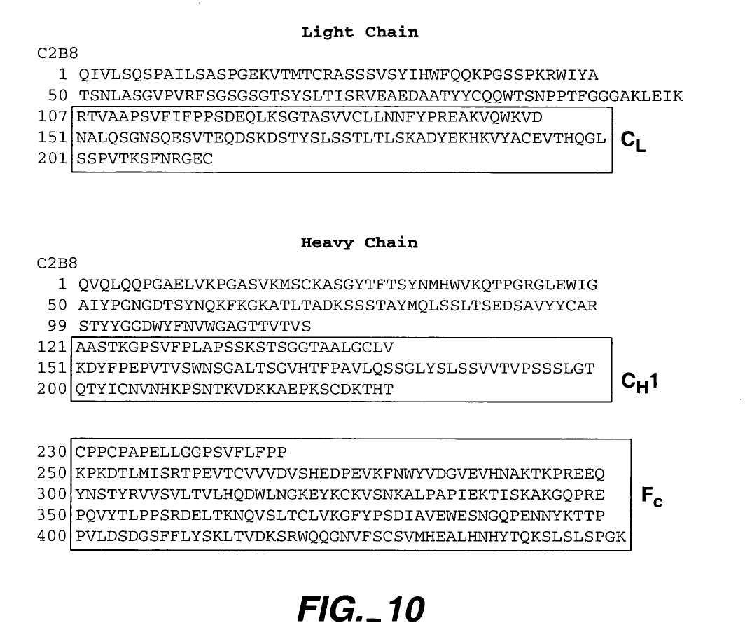

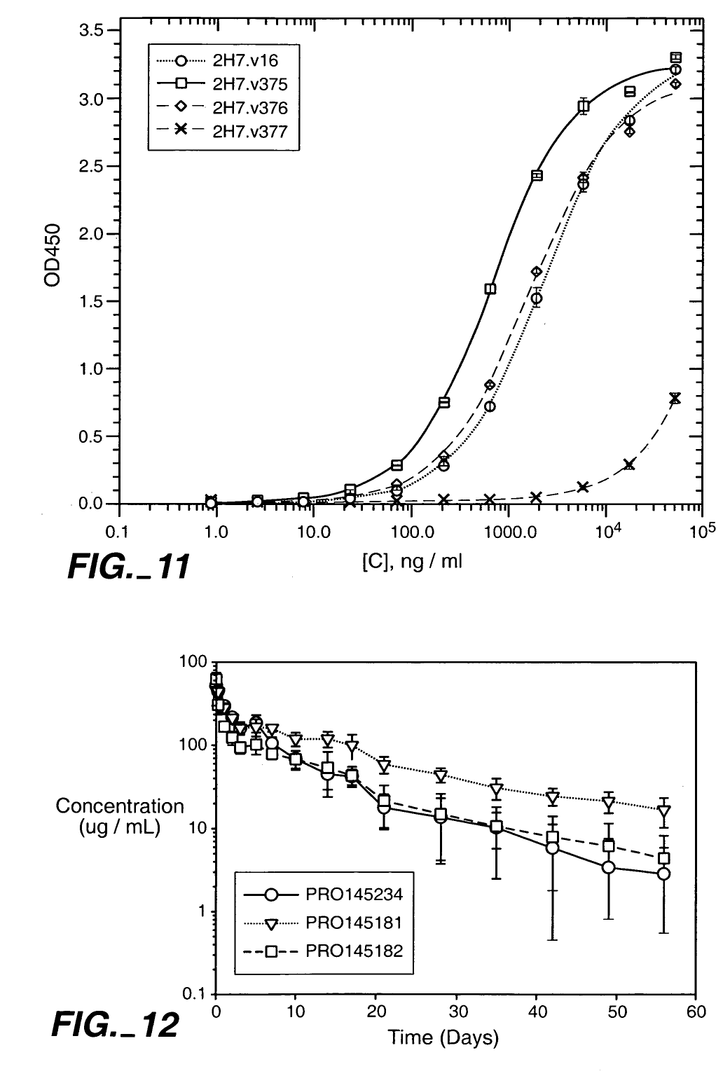

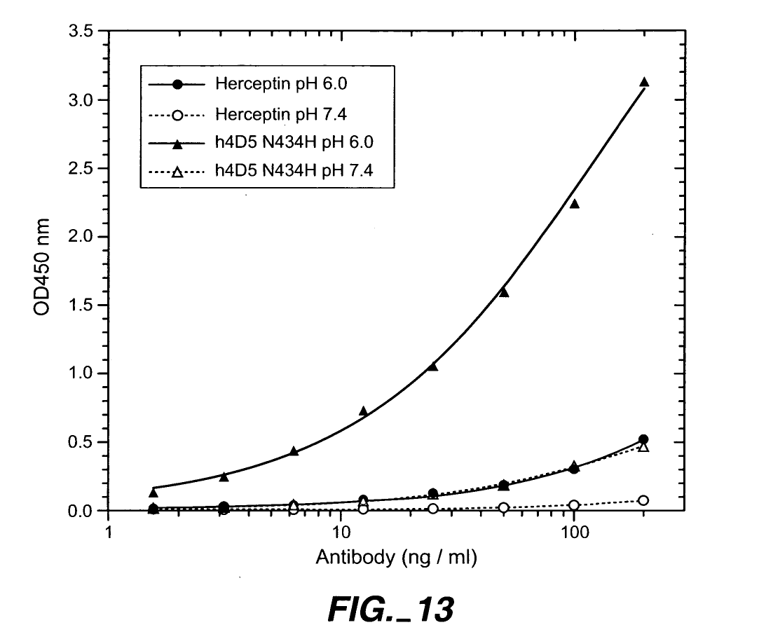

The terms “rituximab” or “RITUXAN®” herein refer to the genetically engineered chimeric murine/human monoclonal antibody directed against the CD20 antigen and designated “C2B8” in U.S. Pat. No. 5,736,137, expressly incorporated herein by reference, including fragments thereof which retain the ability to bind CD20. The C2B8 light (SEQ ID NO.24) and heavy chain (SEQ ID NO.25) sequences are shown in FIG. 10. The V_{L} and V_{H} are delineated.

In specific embodiments, antibodies which bind the CD20 antigen include the humanized 2H7v16 antibody and variants thereof described below. Humanized 2H7v.16 refers to an intact antibody or antibody fragment comprising the variable light sequence:

(SEQ ID NO:1)

DIQMTQSPSSLSASVGDRVTTTCRASSSVSYMHWYQQKPGKAPKPLIYAP
SNLASGVPSRFSGSGSGTDFTLTISSLQPEDFATYYCQQWSFNPPTFGQG
TKVEIKR
and

variable heavy sequence:

(SEQ ID NO: 2)

EVQLVESGGGLVQPGGSLRLSCAASGYTFTSYNMHWVRQAPGKGLEWVGA
IYPGNGDTSYNQKFKGRPTISVDKSKNTLYLQMNSLRAEDTAVYYCARVV
YYSNSYWYFDVWGQGTLVTVSS

Where the humanized 2H7v.16 antibody is an intact antibody, preferably it comprises the v16 full length light chain amino acid sequence:

2H7.v16 Light Chain

(SEQ ID NO: 26)

DIQMTQSPSSLSASVGDRVTTTCRASSSVSYMHWYQQKPGKAPKPLIYAP
SNLASGVPSRFSGSGSGTDFTLTISSLQPEDFATYYCQQWSFNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC;

amino acid sequence:

2H7.v16 Heavy Chain

(SEQ ID NO: 27)

EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSL
SLSPGK.

The V region of all other variants based on version 16 will have the amino acid sequences of v16 except at the positions of amino acid substitutions which are indicated in the table below. Unless otherwise indicated below, the 2H7 variants will have the same L chain as that of v16.

| 2H7 | Heavy chain | Light chain | |
|---|---|---|---|
| version | (V_{H}) changes | (V_{L}) changes | Fc changes |
| 16 | | | — |
| 31 | — | — | S298A, E333A, K334A |
| 73 | N100A | M32L | |
| 75 | N100A | M32L | S298A, E333A, K334A |
| 96 | D56A, N100A | S92A | |
| 114 | D56A, N100A | M32L, S92A | S298A, E333A, K334A |
| 115 | D56A, N100A | M32L, S92A | S298A, E333A, K334A, E356D, |
| | | | M358L |
| 116 | D56A, N100A | M32L, S92A | S298A, K334A, K322A |
| 138 | D56A, N100A | M32L, S92A | S298A, E333A, K334A, K326A |
| 477 | D56A, N100A | M32L, S92A | S298A, E333A, K334A, |
| | | | K326A, N434W |
| 375 | — | — | K334L |

Each of versions 114, 115, 116, 138, 477, 511 comprises the VL sequence:

(SEQ ID NO: 41)

DIQMTQSPSSLSASVGDRVTTTCRASSSVSYLHWYQQKPGKAPKPLIYAP
SNLASGVPSRFSGSGSGTDFTLTISSLQPEDFATYYCQQWAFNPPTFGQG
TKVEIKR

Each of versions 96, 114, 115, 116, 138, 477 comprises the VH sequence:

What is claimed is:

1. An isolated polypeptide comprising a variant IgG Fc region comprising at least an amino acid substitution at Asn 434 to Trp (N434W).

2. The polypeptide of claim 1 wherein the variant Fc binds human FcRn with higher affinity than native sequence IgG Fc region at pH 6.0 and with weaker binding affinity at pH 7.4 than at pH 6.0.

3. The polypeptide of claim 2 wherein the binding affinity for human FcRn at pH 6.0 is at least 20 fold higher than native sequence Fc region.

4. The polypeptide of claim I wherein the polypeptide has a longer serum half life in primate serum than a polypeptide with native sequence Fc region.

5. The polypeptide of claim 4 wherein the primate is human or cynomolgus monkey.

6. The polypeptide of claim 1, wherein the polypeptide is an immunoadhesin.

7. The polypeptide of claim 1 further comprising one or more amino acid substitutions in the Fc region that result in the polypeptide exhibiting at least one of the following properties of increased FcγR binding, increased antibody-dependent cell-mediated cytotoxicity (ADCC), increased complement dependent cytotoxicity (CDC), decreased CDC, increased ADCC and CDC function, increased ADCC but decreased CDC function, increased FcRn binding and serum half life, as compared to an antibody having native sequence Fc region.

8. The polypeptide of claim 1 further comprising one or more amino acid substitutions in the IgG Fc region at a residue position selected from the group consisting of D265A, S298A/E333A/K334A, K334L, K322A, K326A, K326W, E380A and E380A/T307A, wherein the numbering of the residues is that of the EU index as in Kabat.

9. An isolated antibody comprising a variant IgG Fc region comprising at least an amino acid substitution at Asn 434 to Trp (N434W).

10. The antibody of claim 9, wherein the variant IgG Fc binds human FcRn with higher affinity than native sequence IgG Fc region at pH 6.0 and with weaker binding affinity at pH 7.4 than at pH 6.0.

11. The antibody of claim 10, wherein the binding affinity of the variant Fc for human FcRn at pH 6.0 is at least 20 fold higher than that of native sequence IgG Fc region.

12. The antibody of claim 9 wherein the antibody is chimeric, humanized or human.

13. The antibody of claim 12, wherein the antibody is an IgG1.

14. The antibody of claim 9, wherein the antibody further comprises one or more amino acid substitutions in the Fc region that result in the polypeptide exhibiting at least one of the following properties of increased FcγR binding, increased antibody-dependent cell-mediated cytotoxicity (ADCC), increased complement dependent cytotoxicity (CDC), decreased CDC, increased ADCC and CDC function, increased ADCC but decreased CDC function, increased FcRn binding and serum half life, as compared to an antibody having native sequence Fc region.

15. The antibody of claim 9, wherein the antibody further comprises one or more amino acid substitutions in the IgG Fc region at a residue position selected from the group consisting of D265A, S298A/E333A/K334A, K334L, K322A, K326A, K326W, E380A and E380A/T307A, wherein the numbering of the residues is that of the EU index as in Kabat.

16. The antibody of claim 9, wherein the antibody binds an antigen selected from the group of antigens consisting of CD20, Her2, BR3, TNF, VEGF, IgE, and CD11a.

17. The antibody of claim 16, wherein the antibody binds human CD20 and comprises a VH sequence selected from the group of sequences consisting of:
a. SEQ ID NO.2;
b. SEQ ID NO.42; and
c. SEQ ID NO.45
and wherein the L chain comprises the VL sequence of SEQ ID NO.1 or the full length sequence of SEQ ID NO.26.

18. The antibody of claim 16, wherein the antibody binds human CD20 and comprises the C2B8 VL sequence from SEQ ID NO.24 and the VH sequence from SEQ ID NO.25 as shown in FIG. 10.

19. The antibody of claim 16, wherein the antibody binds VEGF and comprises V_{L} and V_{H} sequences selected from the group of sequences consisting of VL sequence of SEQ ID NO.7 with VH sequence of SEQ ID NO.8; VL sequence of SEQ ID NO.9 with VH sequence of SEQ ID NO.10; and VL sequence of SEQ ID NO.11 with VH sequence of SEQ ID NO.12.

20. The antibody of claim 16, wherein the antibody binds Her2 and comprises V_{L} and V_{H} sequences selected from the group of sequences consisting of VL sequence of SEQ ID NO.3 with VH sequence of SEQ ID NO.4; and VL sequence of SEQ ID NO.5 with VH sequence of SEQ ID NO.6.

21. The antibody of claiml6, wherein the antibody binds human CD11a and comprises a VL sequence of SEQ ID NO.13 or the full length L chain of SEQ ID NO.15, with VH sequence of SEQ ID NO.14.

22. The antibody of claim 16, wherein the antibody binds human IgE and comprises V_{L} and V_{H} sequences selected from the group of sequences consisting of VL sequence of SEQ ID NO.47 with VH sequence of SEQ ID NO.48; VL sequence of SEQ ID NO.49 with VH sequence of SEQ ID NO.50; VL sequence of SEQ ID NO.51 with VH sequence of SEQ ID NO.52; VL sequence of SEQ ID NO.53 with VH sequence of SEQ ID NO.54.

23. A composition comprising the polypeptide of claim 1 or the antibody of claim 9, and a carrier.

24. An isolated nucleic acid encoding an antibody of claim 9.

25. An expression vector encoding the polypeptide of claim 1.

26. An isolated host cell comprising a nucleic acid of claim 24.

27. The host cell of claim 26 that produces the antibody of claim 9.

28. A method of producing the antibody of claim 9, comprising culturing the host cell of claim 27 that produces the polypeptide and recovering the polypeptide from the cell culture.

29. An article of manufacture comprising a container and a composition contained therein, wherein the composition comprises a polypeptide of claim 1.

30. The article of manufacture of claim 29, further comprising a package insert indicating that the composition can be used to treat non-Hodgkin's lymphoma.

31. A method of treating a B cell neoplasm or malignancy characterized by B cells expressing CD20, comprising administering to a patient suffering from the neoplasm or malignancy, a therapeutically effective amount of a humanized CD20 binding antibody of claim 17.

32. The method of claim 31, wherein the B cell neoplasm is non-Hodgkin's lymphoma (NHL) or lymphocyte predominant Hodgkin's disease (LPHD).

33. A method of treating chronic lymphocytic leukemia, comprising administering to a patient suffering from the leukemia, a therapeutically effective amount of an antibody of claim 17 which binds human CD20, wherein the antibody further comprises amino acid substitution K326A or K326W.

34. A method of alleviating a B-cell regulated autoimmune disorder, comprising administering to a patient suffering from the disorder, a therapeutically effective amount of a CD20 binding antibody of claims 16 or 17.

35. The method of claim 34, wherein the autoimmune disorder is selected from the group consisting of rheumatoid arthritis, juvenile rheumatoid arthritis, systemic lupus erythematosus (SLE), Wegener's disease, inflammatory bowel disease, idiopathic thrombocytopenic purpura (ITP), thrombotic throbocytopenic purpura (TTP), autoimmune thrombocytopenia, multiple sclerosis, psoriasis, IgA nephropathy, IgM polyneuropathies, myasthenia gravis, vasculitis, diabetes mellitus, Reynaud's syndrome, Sjorgen's syndrome and glomerulonephritis.

36. A method of treating an angiogenesis related disorder, comprising administering to a patient suffering from the disorder, a therapeutically effective amount of an antibody of claim 19.

37. A method of treating a HER2 expressing cancer, comprising administering to a patient suffering from the cancer, a therapeutically effective amount of an antibody of claim 20.

38. A method of treating a LFA-1 mediated disorder, comprising administering to a patient suffering from the disorder, a therapeutically effective amount of an antibody of claim 21.

39. A method of treating an IgE-mediated disorder, comprising administering to a patient suffering from the disorder, a therapeutically effective amount of an antibody of claim 22.

40. An isolated polypeptide comprising a variant IgG Fc region comprising at least an amino acid substitution at Asn 434 to Tyr (N434Y) wherein the polypeptide does not further have an amino acid substitution selected from the group consisting of H433R, H433S, Y436H, Y436R, Y436T.

41. An isolated polypeptide comprising a variant IgG Fc region comprising at least an amino acid substitution at Asn 434 to Phe (N434F) wherein the polypeptide does not further have an amino acid substitution of H433K, Y436H,-M252Y, S254T, or T256E.

42. An isolated polypeptide comprising a variant IgG Fc region comprising at least an amino acid substitution at Asn 434 to His (N434H).

43. The polypeptide of any one of claims 40, 41 and 42, wherein the variant IgG Fc region binds human FcRn with higher affinity than native sequence IgG Fc region at pH 6.0 and with weaker binding affinity at pH 7.4 than at pH 6.0.

44. The polypeptide of any one of claims 40, 41 and 42, wherein the polypeptide is an antibody.

45. The polypeptide of claim 44 wherein the antibody is chimeric, human or humanized.

46. The polypeptide of claim 45 wherein the IgG is human IgG1.

47. The polypeptide of claim 44 wherein the antibody binds an antigen selected from the group of antigens consisting of CD20, HER2, BR3, TNF, VEGF, IgE, and CD11a.

48. The polypeptide of claim 42 which is an antibody that binds HER2.

49. The polypeptide of claim 48, wherein the antibody comprises V_{L} and V_{H} sequences selected from the group of sequences consisting of V_{L} sequence of SEQ ID NO.3 paired with V_{H} sequence of SEQ ID NO.4; and V_{L} sequence of SEQ ID NO.5 paired with V_{H} sequence of SEQ ID NO.6.

50. The polypeptide of claim 48, wherein the HER2 binding antibody further comprises one or more amino acid substitutions in the Fc region that result in the polypeptide exhibiting at least one of the following properties of increased FcγR binding, increased antibody-dependent cell-mediated cytotoxicity (ADCC), increased complement dependent cytotoxicity (CDC), decreased CDC, increased ADCC and CDC function, increased ADCC but decreased CDC function, increased FcRn binding and serum half life, as compared to an antibody having native sequence Fc region.

51. The polypeptide of claim 48 further comprising one or more amino acid substitutions in the IgG Fc region at a residue position selected from the group consisting of D265A, S298A/E333A/K334A, K334L, K322A, K326A, K326W, E380A and E380A/T307A, wherein the numbering of the residues is that of the EU index as in Kabat.

52. The polypeptide of claim 48 further comprising amino acid substitutions T307A/E380A in the IgG Fc region.

53. The polypeptide of claim 52 wherein the antibody comprises V_{L} sequence of SEQ ID NO.5 paired with V_{H} sequence of SEQ ID NO.6. and the binding of the antibody to human FcRn at pH 6.0 is at least 40 fold better than that of parent antibody trastuzumab.

54. The polypeptide of claim 48 further comprising an amino acid substitution of T289H or N315H.

55. The polypeptide of any one of claims 40, 41 and 42, wherein the polypeptide is an immunoadhesin.

56. The polypeptide of any one of claims 40, 41 and 42, further comprising one or more amino acid substitutions in the Fc region that result in the polypeptide exhibiting at least one of the following properties of increased FcγR binding, increased ADCC, increased CDC, decreased CDC, increased ADCC and CDC function, increased ADCC but decreased CDC function.

57. The polypeptide of any one of claims 40, 41 and 42, further comprising one or more amino acid substitutions in the IgG Fc region at a residue position selected from the group consisting of D265A, S298A/E333A/K334A, K334L, K322A, K326A, K326W, E380A and E380A/T307A, wherein the numbering of the residues is that of the EU index as in Kabat.

58. A composition comprising the polypeptide of any one of claims 40, 41, 42, and 48, and a carrier.

59. An isolated nucleic acid encoding a polypeptide of any one of claims 40, 41, 42 and 48.

60. An isolated host cell comprising a nucleic acid of claim 53.

61. The host cell of claim 60 that produces the polypeptide of one any one of claims 40, 41, 42 and 48.

62. A method of producing the polypeptide of claim 42, comprising culturing the host cell of claim 61 that produces the polypeptide of claim 42 and recovering the polypeptide from the cell culture.

63. An article of manufacture comprising a container and a composition contained therein, wherein the composition comprises a polypeptide of any one of claims 40, 41, 42 and 48.

64. A method of treating a HER2 expressing cancer, comprising administering to a patient suffering from the cancer, a therapeutically effective amount of an antibody of claim 48.

65. The method of claim 64, wherein the antibody comprises V_{L} and V_{H} sequences selected from the group of sequences consisting of V_{L} sequence of SEQ ID NO.3 paired with V_{H} sequence of SEQ ID NO.4; and V_{L} sequence of SEQ ID NO.5 paired with V_{H} sequence of SEQ ID NO.6.

66. An isolated polypeptide comprising a variant IgG Fc region comprising at least an amino acid substitution at Lys 334 to Leucine (K334L).

67. The polypeptide of claim 66 wherein the variant Fc binds human FcγRIII with higher affinity than native sequence IgG Fc region.

68. The polypeptide of claim 66 which exhibits increased antibody dependent cytotoxicity in the presence of human effector cells than a polypeptide having native sequence IgG Fc region.

69. The polypeptide of claim 66 further comprising one or more amino acid substitutions in the Fc region that result in the polypeptide exhibiting at least one of the following properties of increased FcγR binding, increased ADCC, increased CDC, decreased CDC, increased ADCC and CDC function, increased ADCC but decreased CDC function, increased FcRn binding and serum half life, as compared to an antibody having the native sequence Fc region.

70. The polypeptide of claim 66 further comprising one or more amino acid substitutions in the IgG Fc region at a residue position selected from the group consisting of D265A, S298A/E333A, K322A, K326A, K326W, E380A and E380A/T307A, wherein the numbering of the residues is that of the EU index as in Kabat.

71. The polypeptide of claim 66 which is a chimeric, humanized or human IgG antibody.

72. A humanized CD20 binding antibody having the L chain sequence of SEQ ID NO.39 and the H chain sequence of SEQ ID NO.40 except that N434 in the Fc region is substituted with W, Y, F or A.

73. A composition comprising the antibody of claim 72 and a carrier.

74. An isolated nucleic acid encoding the antibody of claim 73.

75. A host cell comprising the nucleic acid of claim 74.

76. A method of treating a B cell neoplasm or malignancy characterized by B cells expressing CD20, comprising administering to a patient suffering from the neoplasm or malignancy, a therapeutically effective amount of the humanized CD20 binding antibody of claim 72.

77. A method of alleviating a B-cell regulated autoimmune disorder, comprising administering to a patient suffering from the disorder, a therapeutically effective amount of the humanized CD20 binding antibody of claim 72.

78. A method of screening for a polypeptide with high affinity binding to FcRn at pH 6.0 and with weaker binding affinity at pH 7.4 than at pH 6.0 as compared to a polypeptide with native sequence IgG Fc, the method comprising expressing a candidate polypeptide on phage, providing human FcRn immobilized on a solid matrix, allowing phage particles to bind to the human FcRn on the matrix, removing unbound phage particles by multiple rounds of washes each round with increasing stringency, and eluting the remaining bound phage at pH 7.4.

79. An isolated anti-HER2 antibody comprising V_{L} sequence of SEQ ID NO.5, V_{H} sequence of SEQ ID NO.6, and a variant IgG Fc region comprising at least an amino acid substitution at Asn 434 to Ala (N434A).

80. The antibody of claim 79, further comprising one or more amino acid substitutions in the IgG Fc region at a residue position selected from the group consisting of D265A, S298A/E333A/K334A, K334L, K322A, K326A, K326W, E380A and E380A/T307A, wherein the numbering of the residues is that of the EU index as in Kabat.


SEQUENCE LISTING (54 sequences)

SEQ ID NO: 1 - protein, 107 aa, Artificial sequence
  Sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 2 - protein, 122 aa, Artificial sequence [sequence omitted]

SEQ ID NO: 3 - protein, 107 aa, Artificial sequence [sequence omitted]

SEQ ID NO: 4 - protein, 119 aa, Artificial sequence [sequence omitted]

SEQ ID NO: 5 - protein, 107 aa, Artificial sequence [sequence omitted]

SEQ ID NO: 6 - protein, 120 aa, Artificial Sequence [sequence omitted]

SEQ ID NO: 7 - protein, 108 aa, Artificial Sequence
  Sequence is synthesized
DIQMTQTTSSLSASLGDRVIISCSASQDISNYLNWYQQKPDGTVKVLIYFTSSLHSGVPSRFSGSGSGTDYSLTISNLEPEDIATYYCQQYSTVPWTFGGGTKLEIKR

SEQ ID NO: 8 - protein, 123 aa, Artificial Sequence
  Sequence is synthesized
EIQLVQSGPELKQPGETVRISCKASGYTFTNYGMNWVKQAPGKGLKWMGWINTYTGEPTYAADFKRRFTFSLETSASTAYLQISNLKNDDTATYFCAKYPHYYGSSHWYFDVWGAGTTVTVSS

SEQ ID NO: 9 - protein, 108 aa, Artificial Sequence
  Sequence is synthesized
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 10 - protein, 123 aa, Artificial sequence [sequence omitted]

SEQ ID NO: 11 - protein, 108 aa, Artificial sequence
  Sequence is synthesized
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 12 - protein, 123 aa, Artificial sequence [sequence omitted]

SEQ ID NO: 13 - protein, 108 aa, Artificial sequence
  Sequence is synthesized
DIQMTQSPSSLSASVGDRVTITCRASKTISKYLAWYQQKPGKAPKLLIYSGSTLQSGVPSRFSGSGSGTDFTLTISSLQPEDFATYYCQQHNEYPLTFGQGTKVEIKR

SEQ ID NO: 14 - protein, 121 aa, Artificial sequence [sequence omitted]

SEQ ID NO: 15 - protein, 214 aa, Artificial sequence
  Sequence is synthesized
DIQMTQSPSSLSASVGDRVTITCRASKTISKYLAWYQQKPGKAPKLLIYSGSTLQSGVPSRFSGSGSGTDFTLTISSLQPEDFATYYCQQHNEYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 218 aa, Artificial sequence
  Sequence is synthesized
DIQLTQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 218 aa, Artificial sequence
  Sequence is synthesized
DIQLTQSPSSLSASVGDRVTITCRASKPVDGEGDSYLNWYQQKPGKAPKLLIYAASYLESGVPSRFSGSGSGTDFTLTISSLQPEDFATYYCQQSHEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 218 aa, Artificial sequence; identical to SEQ ID NO: 17
  Sequence is synthesized

SEQ ID NO: 19 - protein, 214 aa, Artificial sequence
  Sequence is synthesized
DILLTQSPGTLSLSPGERATLSCRASQSIGTNIHWYQQKPGQAPRLLIKYASESISGIPSRFSGSGSGTDFTLTISRLEPEDFAMYYCQQSDSWPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 451 aa, Artificial sequence
  Sequence is synthesized
EVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 451 aa, Artificial sequence; identical to SEQ ID NO: 20
  sequence is synthesized

SEQ ID NO: 22 - protein, 451 aa, Artificial sequence
  sequence is synthesized
EVQLVESGGGLVQPGGSLRLSCAVSGYSITSGYSWNWIRQAPGKGLEWVASIKYSGETKYNPSVKGRITISRDDSKNTFYLQMNSLRAEDTAVYYCARGSHYFGHWHFAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 453 aa, Artificial sequence
  sequence is synthesized
QVQLVQSGAEVKKPGASVKVSCKASGYTFSMYWLEWVRQAPGHGLEWVGEISPGTFTTNYNEKFKARATFTADTSTNTAYMELSSLRSEDTAVYYCARFSHFSGSNYDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 213 aa, Artificial sequence
  sequence is synthesized
QIVLSQSPAILSASPGEKVTMTCRASSSVSYIHWFQQKPGSSPKRWIYATSNLASGVPVRFSGSGSGTSYSLTISRVEAEDAATYYCQQWTSNPPTFGGGAKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 451 aa, Artificial sequence
  sequence is synthesized
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 213 aa, Artificial sequence
  sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 218 aa, Homo sapiens
PAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 218 aa, Homo sapiens
PAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 217 aa, Homo sapiens
PAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 32 - protein, 218 aa, Homo sapiens
PAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 33 - protein, 218 aa, Homo sapiens
PAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWZSNGQPENNYKTTPPVLDSDGSFFLYSRLTVDKSRWQEGNVFSCSVMHEALHNHYTQKSLSLSLGK

SEQ ID NO: 34 - protein, 215 aa, Mus musculus
TVPEVSSVFIFPPKPKDVLTITLTPKVTCVVVDISKDDPEVQFSWFVDDVEVHTAQTQPREEQFNSTFRSVSELPIMHQDCLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 35 - protein, 218 aa, Mus musculus
PAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 36 - protein, 218 aa, Mus musculus
PAPNLEGGPSVFIFPPNIKDVLMISLTPKVTCVVVDVSEDDPDVQISWFVNNVEVHTAQTQTHREDYNSTIRVVSHLPIQHQDWMSGKEFKCKVNNKDLPSPIERTISKPKGLVRAPQVYTLPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 37 - protein, 218 aa, Mus musculus
PPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 38 - protein, 220 aa, Homo sapiens
SPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKL

SEQ ID NO: 39 - protein, 213 aa, Artificial sequence
  sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 107 aa, Artificial sequence
  sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKR

SEQ ID NO: 42 - protein, 122 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSS

SEQ ID NO: 43 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 44 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 122 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSS

SEQ ID NO: 46 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 111 aa, Artificial sequence [sequence omitted]

SEQ ID NO: 48 - protein, 121 aa, Artificial sequence [sequence omitted]

SEQ ID NO: 49 - protein, 111 aa, Artificial sequence
  sequence is synthesized
DIQLTQSPSSLSASVGDRVTITCRASKPVDGEGDSYLNWYQQKPGKAPKLLIYAASYLESGVPSRFSGSGSGTDFTLTISSLQPEDFATYYCQQSHEDPYTFGQGTKVEIK

SEQ ID NO: 50 - protein, 121 aa, Artificial sequence [sequence omitted]

SEQ ID NO: 51 - protein, 111 aa, Artificial sequence; identical to SEQ ID NO: 49
  sequence is synthesized

SEQ ID NO: 52 - protein, 121 aa, Artificial sequence
  sequence is synthesized
EVQLVESGGGLVQPGGSLRLSCAVSGYSITSGYSWNWIRQAPGKGLEWVASIKYSGETKYNPSVKGRITISRDDSKNTFYLQMNSLRAEDTAVYYCARGSHYFGHWHFAVWGQGTLVTVSS

SEQ ID NO: 53 - protein, 111 aa, Artificial sequence
  sequence is synthesized
DILLTQSPGTLSLSPGERATLSCRASQSIGTNIHWYQQKPGQAPRLLIKYASESISGIPSRFSGSGSGTDFTLTISRLEPEDFAMYYCQQSDSWPTTFGQGTKVEIKRTVA

SEQ ID NO: 54 - protein, 121 aa, Artificial sequence
  sequence is synthesized
QVQLVQSGAEVKKPGASVKVSCKASGYTFSMYWLEWVRQAPGHGLEWVGEISPGTFTTNYNEKFKARATFTADTSTNTAYMELSSLRSEDTAVYYCARFSHFSGSNYDYFDYWGQGTLVTV